(19) United States
(12) Patent Application Publication
(10) Pub. No.: US 2022/0349904 A1
(43) Pub. Date: Nov. 3, 2022

(54) Cardiovascular Risk Event Prediction and Uses Thereof

(71) Applicant: SomaLogic Operating Co., Inc., Boulder, CO (US)

(72) Inventors: Michael Hinterberg, Boulder, CO (US); Gargi Datta, Boulder, CO (US)

(73) Assignee: SomaLogic Operating Co., Inc., Boulder, CO (US)

(21) Appl. No.: 17/637,128

(22) PCT Filed: Sep. 2, 2020
(86) PCT No.: PCT/US2020/048981

§ 371 Date: Feb. 22, 2022

Related U.S. Application Data
(60) Provisional application No. 62/895,383, filed on Sep. 3, 2019.

(51) Int. Cl. G01N 33/68

(52) U.S. Cl. CPC G01N 33/6893; G01N 33/6872; G01N 2800/50; G01N 2800/32; G01N 2800/324; G01N 2800/60

(57) ABSTRACT

Biomarkers, methods, devices, reagents, systems, and kits used to assess an individual for the prediction of risk of developing a primary or secondary Cardiovascular (CV) event over a 4 year period are provided.

### CROSS-REFERENCE TO RELATED APPLICATIONS

This application claims the benefit of priority of U.S. Provisional Application No. 62/895,383, filed Sep. 3, 2019, which is incorporated by reference herein in its entirety for any purpose.

### FIELD OF THE INVENTION

The present application relates generally to the detection of biomarkers and a method of evaluating the risk of a future cardiovascular event in an individual and, more specifically, to one or more biomarkers, methods, devices, reagents, systems, and kits used to assess an individual for the prediction of risk of developing a primary or secondary Cardiovascular (CV) event over a 4 year period. Such events include but are not limited to myocardial infarction, stroke, trans-ischemic attack, hospitalization for heart failure, and death.

### BACKGROUND

Cardiovascular disease is the leading cause of death in the USA. There are a number of existing and important predictors of risk of primary events (D'Agostino, R et al., “General Cardiovascular Risk Profile for Use in Primary Care: The Framingham Heart Study” Circulation 117:743-53 (2008); and Ridker, P. et al., “Development and Validation of Improved Algorithms for the Assessment of Global Cardiovascular Risk in Women” JAMA 297(6):611-619 (2007)) and secondary events (Shlipak, M. et al. “Biomarkers to Predict Recurrent Cardiovascular Disease: The Heart & Soul Study” Am. J. Med. 121:50-57 (2008)) which are widely used in clinical practice and therapeutic trials. Unfortunately, the receiver-operating characteristic curves, hazard ratios, and concordance show that the performance of existing risk factors and biomarkers is modest (AUCs of ˜0.75 mean that these factors are only halfway between a coin-flip and perfection). In addition to a need for improved diagnostic performance, there is a need for a risk product which is both near-term and personally responsive within individuals to beneficial (and destructive) interventions and lifestyle changes. The commonly utilized Framingham equation has three main problems. Firstly, it is too long term: it gives 10-year risk calculations but humans discount future risks and are reluctant to make behavior and lifestyle modifications based on them. Secondly, it is not very responsive to interventions: it is heavily dependent on chronological age, which cannot decline; and gender, which cannot change. Thirdly, within the high risk population envisioned here, the Framingham factors fail to discriminate well between high and low risk: the hazard ratio between high and low quartiles is only 2, and when one attempts to use Framingham scores to personalize risk by stratifying subjects into finer layers (deciles for example) the observed event rates are similar for many of the deciles.

Risk factors for cardiovascular disease are widely used to drive the intensity and the nature of medical treatment, and their use has undoubtedly contributed to the reduction in cardiovascular morbidity and mortality that has been observed over the past two decades. These factors have routinely been combined into algorithms but unfortunately they do not capture all of the risk (the most common initial presentation for heart disease is still death). In fact they probably only capture half the risk. An area under the ROC curve of ˜0.76 is typical for such risk factors in primary prevention, with much worse performance in secondary prevention (0.62 is typical), numbers only about one quarter to one half of the performance between a coin-flip at 0.5 and perfection at 1.0.

Moreover, in the Framingham study (Wang et al., “Multiple Biomarkers for the Prediction of First Major Cardiovascular Events and Death” N. Eng. J. Med. 355:2631-2637 (2006)) in 3209 people, the addition of 10 biomarkers (CRP, BNP, NT-proBNP, aldosterone, renin, fibrinogen, D-dimer, plasminogen-activator inhibitor type 1, homocysteine and the urinary albumin to creatinine ratio), did not significantly improve the AUC when added to existing risk factors: the AUC for events 0-5 years was 0.76 with age, sex and conventional risk factors and 0.77 with the best combination of biomarkers added to the mix, and for secondary prevention the situation is worse.

Early identification of patients with higher risk of a cardiovascular event within a 1-5 year window is important because more aggressive treatment of individuals with elevated risk may improve outcome. Thus, optimal management requires aggressive intervention to reduce the risk of a cardiovascular event in those patients who are considered to have a higher risk, while patients with a lower risk of a cardiovascular event can be spared expensive and potentially invasive treatments, which are likely to have no beneficial effect to the patient.

Biomarker selection for the prediction of risk of having specific disease state or condition within a defined time period involves first the identification of markers that have a measurable and statistically significant relationship with the probability and/or timing of an event for a specific medical application. Biomarkers can include secreted or shed molecules that are either on the causal pathway to the condition of interest, or which are downstream or parallel to the disease or condition development or progression, or both. They are released into the blood stream from cardiovascular tissue or from other organs and surrounding tissues and circulating cells in response to the biological processes which predispose to a cardiovascular event or they may be reflective of downstream effects of the pathophysiology such as a decline in kidney function. Biomarkers can include small molecules, peptides, proteins, and nucleic acids. Some of the key issues that affect the identification of biomarkers include over-fitting of the available data and bias in the data.

A variety of methods have been utilized in an attempt to identify biomarkers and diagnose or predict the risk of having disease or a condition. For protein-based markers, these include two-dimensional electrophoresis, mass spectrometry, and immunoassay methods. For nucleic acid markers, these include mRNA expression profiles, microRNA profiles, FISH, serial analysis of gene expression (SAGE), large scale gene expression arrays, gene sequencing and genotyping (SNP or small variant analysis).

The utility of two-dimensional electrophoresis is limited by low detection sensitivity; issues with protein solubility, charge, and hydrophobicity; gel reproducibility; and the possibility of a single spot representing multiple proteins. For mass spectrometry, depending on the format used, limitations revolve around the sample processing and separation, sensitivity to low abundance proteins, signal to noise considerations, and inability to immediately identify the detected protein. Limitations in immunoassay approaches to biomarker discovery are centered on the inability of antibody-based multiplex assays to measure a large number of analytes. One might simply print an array of high-quality antibodies and, without sandwiches, measure the analytes bound to those antibodies. (This would be the formal equivalent of using a whole genome of nucleic acid sequences to measure by hybridization all DNA or RNA sequences in an organism or a cell. The hybridization experiment works because hybridization can be a stringent test for identity.) However, even very good antibodies are typically not stringent enough in selecting their binding partners to work in the context of blood or even cell extracts because the protein ensemble in those matrices have widely varying abundances, which can lead to poor signal to noise ratios. Thus, one must use a different approach with immunoassay-based approaches to biomarker discovery—one would need to use multiplexed ELISA assays (that is, sandwiches) to get sufficient stringency to measure many analytes simultaneously to decide which analytes are indeed biomarkers. Sandwich immunoassays do not scale to high content, and thus biomarker discovery using stringent sandwich immunoassays is not possible using standard array formats. Lastly, antibody reagents are subject to substantial lot variability and reagent instability. The instant platform for protein biomarker discovery overcomes this problem.

Many of these methods rely on or require some type of sample fractionation prior to the analysis. Thus the sample preparation required to run a sufficiently powered study designed to identify and discover statistically relevant biomarkers in a series of well-defined sample populations is extremely difficult, costly, and time consuming. During fractionation, a wide range of variability can be introduced into the various samples. For example, a potential marker could be unstable to the process, the concentration of the marker could be changed, inappropriate aggregation or disaggregation could occur, and inadvertent sample contamination could occur and thus obscure the subtle changes anticipated in early disease.

It is widely accepted that biomarker discovery and detection methods using these technologies have serious limitations for the identification of diagnostic or predictive biomarkers. These limitations include an inability to detect low-abundance biomarkers, an inability to consistently cover the entire dynamic range of the proteome, irreproducibility in sample processing and fractionation, and overall irreproducibility and lack of robustness of the method. Further, these studies have introduced biases into the data and not adequately addressed the complexity of the sample populations, including appropriate controls, in terms of the distribution and randomization required to identify and validate biomarkers within a target disease population.

Although efforts aimed at the discovery of new and effective biomarkers have gone on for several decades, the efforts have been largely unsuccessful. Biomarkers for various diseases typically have been identified in academic laboratories, usually through an accidental discovery while doing basic research on some disease process. Based on the discovery and with small amounts of clinical data, papers were published that suggested the identification of a new biomarker. Most of these proposed biomarkers, however, have not been confirmed as real or useful biomarkers, primarily because the small number of clinical samples tested provide only weak statistical proof that an effective biomarker has in fact been found. That is, the initial identification was not rigorous with respect to the basic elements of statistics.

Based on the history of failed biomarker discovery efforts, theories have been proposed that further promote the general understanding that biomarkers for diagnosis, prognosis or prediction of risk of developing diseases and conditions are rare and difficult to find. Biomarker research based on 2D gels or mass spectrometry supports these notions. Very few useful biomarkers have been identified through these approaches. However, it is usually overlooked that 2D gel and mass spectrometry measure proteins that are present in blood at approximately 1 nM concentrations and higher, and that this ensemble of proteins may well be the least likely to change with disease or the development of a particular condition. Other than the instant biomarker discovery platform, proteomic biomarker discovery platforms that are able to accurately measure protein expression levels at much lower concentrations do not exist.

Much is known about biochemical pathways for complex human biology. Many biochemical pathways culminate in or are started by secreted proteins that work locally within the pathology; for example, growth factors are secreted to stimulate the replication of other cells in the pathology, and other factors are secreted to ward off the immune system, and so on. While many of these secreted proteins work in a paracrine fashion, some operate distally in the body. One skilled in the art with a basic understanding of biochemical pathways would understand that many pathology-specific proteins ought to exist in blood at concentrations below (even far below) the detection limits of 2D gels and mass spectrometry. What must precede the identification of this relatively abundant number of disease biomarkers is a proteomic platform that can analyze proteins at concentrations below those detectable by 2D gels or mass spectrometry.

As is discussed above, cardiovascular events may be prevented by aggressive treatment if the propensity for such events can be accurately determined, and by targeting such interventions at the people who need them the most and/or away from people who need them the least, medical resourcing efficiency can be improved and costs may be lowered at the same time. Additionally, when the patient has the knowledge of accurate and near-term information about their personal likelihood of cardiovascular events, this is less deniable than long-term population-based information and will lead to improved lifestyle choices and improved compliance with medication which will add to the benefits. Existing multi-marker tests either require the collection of multiple samples from an individual or require that a sample be partitioned between multiple assays. Optimally, an improved test would require only a single blood, urine or other sample, and a single assay. Accordingly, a need exists for biomarkers, methods, devices, reagents, systems, and kits that enable the prediction of cardiovascular events within a 5 year period.

### SUMMARY OF THE INVENTION

The present application includes biomarkers, methods, reagents, devices, systems, and kits for the prediction of risk of having a Cardiovascular (CV) event, e.g., within a 4 year period or other period of time. In some embodiments, the CV event is a primary CV event. In some embodiments, the CV event is a secondary CV event.

Cardiovascular disease involves multiple biological processes and tissues. Examples of biological systems and processes associated with cardiovascular disease are inflammation, thrombosis, disease-associated angiogenesis, platelet activation, macrophage activation, liver acute response, extracellular matrix remodeling, and renal function. These processes can be observed as a function of gender, menopausal status, and age, and according to status of coagulation and vascular function. Since these systems communicate partially through protein based signaling systems, and multiple proteins may be measured in a single blood sample, the invention provides a single sample, single assay multiple protein based test focused on proteins from the specific biological systems and processes involved in cardiovascular disease.

In some embodiments, methods of detecting levels of a set of biomarkers are provided. In some embodiments, such methods are the following:
Embodiment 1. A method of detecting levels of a set of biomarker proteins in a sample from a subject, comprising:
   a. contacting the sample from the subject with a set of capture reagents, wherein each capture reagent specifically binds to a different biomarker protein, wherein one capture reagent specifically binds to sTREM1; and
   b. detecting the amount of each capture reagent bound to the biomarker protein to which it specifically binds.
Embodiment 2. The method of embodiment 1, wherein one capture reagent specifically binds to MMP-12.
Embodiment 3. The method of embodiment 1 or 2, wherein one capture reagent specifically binds to N-terminal pro-BNP.
Embodiment 4. The method of any one of embodiments 1-3, wherein one capture reagent specifically binds to Antithrombin III.
Embodiment 5. The method of any one of embodiments 1-4, wherein one capture reagent specifically binds to GPR56.
Embodiment 6. The method of any one of embodiments 1-5, wherein one capture reagent specifically binds to Gelsolin.
Embodiment 7. The method of any one of embodiments 1-6, wherein one capture reagent specifically binds to ST4S6.
Embodiment 8. The method of any one of embodiments 1-7, wherein one capture reagent specifically binds to CHSTC.
Embodiment 9. The method of any one of embodiments 1-8, wherein one capture reagent specifically binds to FSH.
Embodiment 10. The method of any one of embodiments 1-9, wherein one capture reagent specifically binds to IL-1 sRII.
Embodiment 11. The method of any one of embodiments 1-10, wherein one capture reagent specifically binds to PLXB2.
Embodiment 12. The method of any one of embodiments 1-11, wherein one capture reagent specifically binds to SAP.
Embodiment 13. The method of any one of embodiments 1-12, wherein one capture reagent specifically binds to TFPI.
Embodiment 14. The method of any one of embodiments 1-13, wherein the set of biomarkers comprises at least three biomarkers.
Embodiment 15. The method of any one of embodiments 1-13, wherein the set of biomarkers comprises at at least 4, at least 5, at least 6, at least 7, at least 8, at least 9, at least 10, at least 11, at least 12, or at least 13 biomarkers.
Embodiment 16. The method of any one of embodiments 1-13, wherein the set of biomarkers consists of 2-13 biomarkers.
Embodiment 17. The method of any one of embodiments 1-13, wherein the set of biomarkers comprises at least 13 biomarkers.
Embodiment 18. The method of any one of embodiments 1-13, wherein the set of biomarkers consists of 13 biomarkers.
Embodiment 19. The method of any one of embodiments 1-18, wherein the subject is at least 40 years old.
Embodiment 20. The method of any one of embodiments 1-19, wherein the subject has no known history of cardiovascular disease.
Embodiment 21. The method of any one of embodiments 1-20, comprising determining the risk of the subject having a primary cardiovascular event within four years from the date the sample was taken from the subject.
Embodiment 22. The method of embodiment 21, wherein the risk of the subject having a primary cardiovascular event is within one, two, three, or four years from the date that sample was taken from the subject.
Embodiment 23. The method of embodiment 21 or 22, wherein the primary cardiovascular event is myocardial infarction, stroke, trans-ischemic attack, hospitalization for heart failure, or death due to cardiovascular disease.
Embodiment 24. The method of any one of embodiments 21-23, wherein the risk is determined as a quantitative probability.
Embodiment 25. The method of any one of embodiments 21-23, wherein the risk is determined as a qualitative level of risk.
Embodiment 26. The method of embodiment 25, wherein the qualitative level of risk is low, moderate, or high.
Embodiment 27. The method of embodiment 1 or 2, wherein one capture reagent specifically binds to SVEP1.
Embodiment 28. The method of anyone of embodiments 1, 2, or 27, wherein one capture reagent specifically binds to ARL11.
Embodiment 29. The method of any one of embodiments 1, 2, 27, or 28, wherein one capture reagent specifically binds to ANTR2.
Embodiment 30. The method of any one of embodiments 1, 2, or 27-29, wherein one capture reagent specifically binds to CA125.
Embodiment 31. The method of any one of embodiments 1, 2, or 27-30, wherein one capture reagent specifically binds to GOLM1.
Embodiment 32. The method of any one of embodiments 1, 2, or 27-31, wherein one capture reagent specifically binds to PPR1A.
Embodiment 33. The method of any one of embodiments 1, 2, or 27-32, wherein one capture reagent specifically binds to ERBB3.
Embodiment 34. The method of any one of embodiments 1, 2, or 27-33, wherein one capture reagent specifically binds to suPAR.
Embodiment 35. The method of any one of embodiments 1, 2, or 27-34, wherein one capture reagent specifically binds to GDF-11/8.
Embodiment 36. The method of any one of embodiments 1, 2, or 27-35, wherein one capture reagent specifically binds to JAM-B.
Embodiment 37. The method of any one of embodiments 1, 2, or 27-36, wherein one capture reagent specifically binds to ATS13.
Embodiment 38. The method of any one of embodiments 1, 2, or 27-37, wherein one capture reagent specifically binds to Spondin-1.
Embodiment 39. The method of any one of embodiments 1, 2, or 27-38, wherein one capture reagent specifically binds to NCAM-120.
Embodiment 40. The method of any one of embodiments 1, 2, or 27-39, wherein one capture reagent specifically binds to TFF3.
Embodiment 41. The method of any one of embodiments 1, 2, or 27-40, wherein one capture reagent specifically binds to SIRT2.
Embodiment 42. The method of any one of embodiments 1, 2, or 27-41, wherein one capture reagent specifically binds to ANP.
Embodiment 43. The method of any one of embodiments 1, 2, or 27-42, wherein one capture reagent specifically binds to NELL1.
Embodiment 44. The method of any one of embodiments 1, 2, or 27-43, wherein one capture reagent specifically binds to LRP11.
Embodiment 45. The method of any one of embodiments 1, 2, or 27-44, wherein one capture reagent specifically binds to NDST1.
Embodiment 46. The method of any one of embodiments 1, 2, or 27-45, wherein one capture reagent specifically binds to PTPRJ.
Embodiment 47. The method of any one of embodiments 1, 2, or 27-46, wherein one capture reagent specifically binds to CILP2.
Embodiment 48. The method of any one of embodiments 1, 2, or 27-47, wherein one capture reagent specifically binds CA2D3.
Embodiment 49. The method of any one of embodiments 1, 2, or 27-48, wherein one capture reagent specifically binds to ITI heavy chain H2.
Embodiment 50. The method of any one of embodiments 1, 2, or 27-49, wherein one capture reagent specifically binds to IGDC4.
Embodiment 51. The method of any one of embodiments 1, 2, or 27-50, wherein one capture reagent specifically binds to BNP.
Embodiment 52. The method of any one of embodiments 27-51, wherein the set of biomarkers comprises at least three biomarkers.
Embodiment 53. The method of any one of embodiments 1, 2, or 27-51, wherein the set of biomarkers comprises at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, or at least 27 biomarkers.
Embodiment 54. The method of any one of embodiments 1, 2, or 27-51, wherein the set of biomarkers consists of 2-27 biomarkers.
Embodiment 55. The method of any one of embodiments 1, 2, or 27-51, wherein the set of biomarkers comprises at least 27 biomarkers.
Embodiment 56. The method of any one of embodiments 1, 2, or 27-51, wherein the set of biomarkers consists of 27 biomarkers.
Embodiment 57. The method of any one of embodiments 27-56, wherein the subject is at least 40 years old.
Embodiment 58. The method of any one of embodiments 27-57, wherein the subject has apparently stable cardiovascular disease.
Embodiment 59. The method of embodiment 58, wherein the apparently stable cardiovascular disease comprises a history of a myocardial infarction, stroke, heart failure, revascularization, abnormal stress test, imaging suggesting coronary heart disease, or abnormal coronary calcium score.
Embodiment 60. The method of embodiment 59, wherein the myocardial infarction or stroke occurred at least six months prior to the date the sample was taken from the subject.
Embodiment 61. The method of embodiment 59, wherein the abnormal stress test is a treadmill or nuclear medicine based test.
Embodiment 62. The method of embodiment 59, wherein the imaging suggesting coronary heart disease is an angiogram showing coronary artery stenosis of 50% or greater.
Embodiment 63. The method of any one of embodiments 27-62, comprising determining the risk of the subject having a secondary cardiovascular event within four years from the date the sample was taken from the subject.
Embodiment 64. The method of embodiment 63, wherein the risk of the subject having a secondary cardiovascular event is within one, two, three, or four years from the date that the sample was taken from the subject.
Embodiment 65. The method of embodiment 63 or 64, wherein the secondary cardiovascular event is myocardial infarction, stroke, trans-ischemic attack, hospitalization for heart failure, or death.
Embodiment 66. The method of any one of embodiments 63-65, wherein the risk is determined as a quantitative probability.
Embodiment 67. The method of any one of embodiments 63-65, wherein the risk is determined as a qualitative level of risk.
Embodiment 68. The method of embodiment 67, wherein the qualitative level of risk is low, moderate, or high.

In some embodiments, methods for screening a subject for the risk of a cardiovascular event (CV) event are provided. In some such embodiments, a method comprises
(a) forming a biomarker panel comprising N biomarkers selected from N-terminal pro-BNP, sTREM-1, MMP-12, Antithrombin III, GPR56, Gelsolin, ST4S6, CHSTC, FSH, IL-1 sRII, PLXB2, SAP, and TFPI, wherein N is an integer from 3 to 13; and
(b) detecting the level of each of the N biomarkers of the panel in a sample from the subject.

In some embodiments, a method comprises
(a) forming a biomarker panel comprising N protein biomarkers selected from BNP, sTREM-1, MMP-12, SVEP1, ARL11, ANTR2, CA125, GOLM1, PPR1A, ERBB3, suPAR, GDF-11/8, JAM-B, ATS13, Spondin-1, NCAM-120, TFF3, SIRT2, ANP, NELL1, LRP11, NDST1, PTPRJ, CILP2, CA2D3, ITI heavy chain H2, and IGDC4, wherein N is an integer from 8 to 27; and
(b) detecting the level of each of the N biomarkers of the panel in a sample from the subject.

In some embodiments, methods for predicting the likelihood that a subject will have a CV event are provided. In some such embodiments, a method comprises
(a) forming a biomarker panel comprising N biomarkers selected from N-terminal pro-BNP, sTREM-1, MMP-12, Antithrombin III, GPR56, Gelsolin, ST4S6, CHSTC, FSH, IL-1 sRII, PLXB2, SAP, and TFPI, wherein N is an integer from 3 to 13; and
(b) detecting the level of each of the N biomarkers of the panel in a sample from the subject.

In some embodiments, a method comprises
(a) forming a biomarker panel comprising N protein biomarkers selected from BNP, sTREM-1, MMP-12, SVEP1, ARL11, ANTR2, CA125, GOLM1, PPR1A, ERBB3, suPAR, GDF-11/8, JAM-B, ATS13, Spondin-1, NCAM-120, TFF3, SIRT2, ANP, NELL1, LRP11, NDST1, PTPRJ, CILP2, CA2D3, ITI heavy chain H2, and IGDC4, wherein N is an integer from 8 to 27; and
(b) detecting the level of each of the N biomarkers of the panel in a sample from the subject.

In some embodiments, methods for screening a subject for the risk or likelihood of a cardiovascular event (CV) event are provided, comprising detecting the level of at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, or 27 of the biomarkers in the panel comprising N biomarkers.

In some embodiments, the risk or likelihood of the subject having a CV event within 4 years is high if the levels of at least 5, at least 6, at least 7, at least 8, at least 9, at least 10, at least 11, at least 12, or at least 13 biomarkers of the set of biomarkers are each abnormal relative to a control level of the respective biomarker.

In some embodiments, the method comprises detecting the level of one or more biomarkers in Table 1. In some embodiments, the method comprises detecting the level of one or more biomarkers in Table 2.

In some embodiments, the subject has coronary artery disease. In some embodiments, the subject does not have a history of CV events. In some embodiments, the subject has a high risk classification in the American College of Cardiology (ACC) pooled cohort equation (PCE). See Goff D C, Jr. et al., “ACC/AHA Guideline on the Assessment of Cardiovascular Risk: A Report of the American College of Cardiology/American Heart Association Task Force on Practice Guidelines.” Circulation. 2013. In some embodiments, the subject has an intermediate risk classification in the PCE. In some embodiments, the subject has a low risk classification in the PCE. In some embodiments, the subject has had at least one CV event. In some embodiments, the CV event is selected from myocardial infarction, stroke, hospitalization for heart failure, trans-ischemic attack, and death.

In some embodiments, the sample is selected from a blood sample, a serum sample, a plasma sample, and a urine sample. In some embodiments, the sample is a plasma sample. In some embodiments, the method is performed in vitro.

In some embodiments, each biomarker is a protein biomarker. In some embodiments, the method comprises contacting biomarkers of the sample from the subject with a set of biomarker capture reagents, wherein each biomarker capture reagent of the set of biomarker capture reagents specifically binds to a different biomarker being detected. In some embodiments, each biomarker capture reagent is an antibody or an aptamer. In some embodiments, each biomarker capture reagent is an aptamer. In some embodiments, at least one aptamer is a slow off-rate aptamer. In some embodiments, at least one slow off-rate aptamer comprises at least one, at least two, at least three, at least four, at least five, at least six, at least seven, at least eight, at least nine, or at least 10 nucleotides with modifications. In some embodiments, each slow off-rate aptamer binds to its target protein with an off rate (t_{1/2}) of ≥30 minutes, ≥60 minutes, ≥90 minutes, ≥120 minutes, ≥150 minutes, ≥180 minutes, ≥210 minutes, or ≥240 minutes.

In some embodiments, the risk or likelihood of a CV event is based on the biomarker levels and at least one item of additional biomedical information selected from
a) information corresponding to the presence of cardiovascular risk factors selected from the group consisting of prior myocardial infarction, angiographic evidence of greater than 50% stenosis in one or more coronary vessels, exercise-induced ischemia by treadmill or nuclear testing or prior coronary revascularization,
b) information corresponding to physical descriptors of the subject,
c) information corresponding to a change in weight of the subject,
d) information corresponding to the ethnicity of the subject,
e) information corresponding to the gender of the subject,
f) information corresponding to the subject's smoking history,
g) information corresponding to the subject's alcohol use history,
h) information corresponding to the subject's occupational history,
i) information corresponding to the subject's family history of cardiovascular disease or other circulatory system conditions,
j) information corresponding to the presence or absence in the subject of at least one genetic marker correlating with a higher risk of cardiovascular disease in the subject or a family member of the subject,
k) information corresponding to clinical symptoms of the subject,
l) information corresponding to other laboratory tests,
m) information corresponding to gene expression values of the subject, and
n) information corresponding to the subject's consumption of known cardiovascular risk factors such as diet high in saturated fats, high salt, high cholesterol,
o) information corresponding to the subject's imaging results obtained by techniques selected from the group consisting of electrocardiogram, echocardiography, carotid ultrasound for intima-media thickness, flow mediated dilation, pulse wave velocity, ankle-brachial index, stress echocardiography, myocardial perfusion imaging, coronary calcium by CT, high resolution CT angiography, MRI imaging, and other imaging modalities,
p) information regarding the subject's medications
q) information corresponding to the age of the subject, and
r) information regarding the subject's kidney function.

In some embodiments, the risk or likelihood of a CV event is based on the biomarker levels and at least the age of the subject.

In some embodiments, the method comprises determining the risk or likelihood of a CV event for the purpose of determining a medical insurance premium or life insurance premium. In some embodiments, the method further comprises determining coverage or premium for medical insurance or life insurance. In some embodiments, the method further comprises using information resulting from the method to predict and/or manage the utilization of medical resources. In some embodiments, the method further comprises using information resulting from the method to enable a decision to acquire or purchase a medical practice, hospital, or company.

In some embodiments, a computer-implemented method for evaluating the risk or likelihood of a cardiovascular (CV) event is provided. In some embodiments, the method comprises retrieving on a computer biomarker information for a subject, wherein the biomarker information comprises (a) the levels of 3 to 13 biomarkers selected from Table 1 in a sample from the subject; or (b) the levels of 8 to 27 biomarkers selected from Table 2; performing with the computer a classification of each of said biomarker values; indicating a result of the evaluation of risk for a CV event for the subject based upon a plurality of classifications. In some embodiments, indicating the result of the evaluation of the risk or likelihood of a CV event for the subject comprises displaying the result on a computer display.

### BRIEF DESCRIPTION OF THE DRAWINGS

FIG. 1 shows Kaplan-Meier survival curves, stratified by the four risk bins of the primary CVD model, for the HUNT3 training set, with shaded regions representing 95% confidence intervals of the Kaplan-Meier estimates. The lines are, from top to bottom, 1× (<0.0215), n=469; 2×-3× (<0.0505), n=944; 4×-5× (<0.077), n=285; 6× and above (>0.077), n=315.

FIG. 2 shows Kaplan-Meier survival curves, stratified by the four risk bins of the secondary CVD model, for the HUNT3 training set, with shaded regions representing 95% confidence intervals of the Kaplan-Meier estimates. The lines are, from top to bottom, <0.075, n=117; <0.25, n=285; >0.5, n=121; >0.5, n=82.

FIG. 3 shows Kaplan-Meier survival curves, stratified by the four risk bins of the secondary CVD model, for the ARIC visit 5 verification set, with shaded regions representing 95% confidence intervals of the Kaplan-Meier estimates. The lines are, from top to bottom, <0.075, n=35; <0.25, n=103; <0.5, n=43; >0.5, n=27.

FIG. 4 shows the survival curves for the HUNT3 validation set, stratified by cutoffs. The lines are, from top to bottom, <0.075, n=24; <0.25, n=61; <0.5, n=25; >0.5, n=29.

FIG. 5 shows the survival curves for the ARIC visit 5 validation set, stratified by cutoffs. The lines are, from top to bottom, <0.075, n=13; <0.25, n=202; <0.5, n=271; >0.5, n=345.

FIG. 6 illustrates a nonlimiting exemplary computer system for use with various computer-implemented methods described herein.

FIG. 7 illustrates a nonlimiting exemplary aptamer assay that can be used to detect one or more biomarkers in a biological sample.

FIG. 8A and FIG. 8B show certain exemplary modified pyrimidines that may be incorporated into aptamers, such as slow off-rate aptamers.

### DETAILED DESCRIPTION

While the invention will be described in conjunction with certain representative embodiments, it will be understood that the invention is defined by the claims, and is not limited to those embodiments.

One skilled in the art will recognize many methods and materials similar or equivalent to those described herein may be used in the practice of the present invention. The present invention is in no way limited to the methods and materials described.

Unless defined otherwise, technical and scientific terms used herein have the meaning commonly understood by one of ordinary skill in the art to which this invention belongs. Although any methods, devices, and materials similar or equivalent to those described herein can be used in the practice of the invention, certain methods, devices, and materials are described herein.

All publications, published patent documents, and patent applications cited herein are hereby incorporated by reference to the same extent as though each individual publication, published patent document, or patent application was specifically and individually indicated as being incorporated by reference.

As used herein, the terms “comprises,” “comprising,” “includes,” “including,” “contains,” “containing,” and any variations thereof, are intended to cover a non-exclusive inclusion, such that a process, method, product-by-process, or composition of matter that comprises, includes, or contains an element or list of elements may include other elements not expressly listed.

The present application includes biomarkers, methods, devices, reagents, systems, and kits for the prediction of risk of near-term CV events within a defined period of time, such as within 1 year, within 2 years, within 3 years, or within 4 years.

“Cardiovascular event” or “CV event” means a failure or malfunction of any part of the circulatory system. In some embodiments, “Cardiovascular event” means stroke, trans-ischemic attack (TIA), myocardial infarction (MI), sudden death attributable to malfunction of the circulatory system, and/or hospitalization for heart failure, or sudden death of unknown cause in a population where the most likely cause is cardiovascular. A primary CV event is the first CV event experienced by a subject. A secondary CV event is the second or further CV event experienced by a subject.

Cardiovascular events may include thrombotic events, such as MIs, trans-ischemic attacks (TIA), stroke, acute coronary syndrome and need for coronary re-vascularization.

In some embodiments, biomarkers are provided for use either alone or in various combinations to evaluate the risk or likelihood of sudden death or a future CV event within a 4 year time period with CV events defined as myocardial infarction, stroke, trans-ischemic attack, death and hospitalization for heart failure. As described in detail below, exemplary embodiments include the biomarkers provided in Table 1 or in Table 2.

While certain of the described CV event biomarkers may be useful alone for evaluating the risk or likelihood of a CV event, methods are also described herein for the grouping of multiple subsets of the CV event biomarkers, where each grouping or subset selection is useful as a panel of three or more biomarkers, interchangeably referred to herein as a “biomarker panel” and a panel. Thus, various embodiments provide combinations comprising at least four, at least five, art least six, at least seven, at least eight, at least nine, at least ten, at least eleven, at least twelve or all thirteen of the biomarkers in Table 1. Other various embodiments provide combinations comprising at least eight, at least nine, at least ten, at least eleven, at least twelve, at least thirteen, at least fourteen, at least fifteen, at least sixteen, at least seventeen, at least eighteen, at least nineteen, at least twenty, at least twenty-one, at least twenty-two, at least twenty-three, at least twenty-four, at least twenty-five, at least twenty-six, or all twenty-seven of the biomarkers in Table 2.

“Biological sample”, “sample”, and “test sample” are used interchangeably herein to refer to any material, biological fluid, tissue, or cell obtained or otherwise derived from an individual. This includes blood (including whole blood, leukocytes, peripheral blood mononuclear cells, buffy coat, plasma, and serum), sputum, tears, mucus, nasal washes, nasal aspirate, urine, saliva, peritoneal washings, ascites, cystic fluid, glandular fluid, lymph fluid, bronchial aspirate, synovial fluid, joint aspirate, organ secretions, cells, a cellular extract, and cerebrospinal fluid. This also includes experimentally separated fractions of all of the preceding. For example, a blood sample can be fractionated into serum, plasma, or into fractions containing particular types of blood cells, such as red blood cells or white blood cells (leukocytes). In some embodiments, a blood sample is a dried blood spot. In some embodiments, a plasma sample is a dried plasma spot. In some embodiments, a sample can be a combination of samples from an individual, such as a combination of a tissue and fluid sample. The term “biological sample” also includes materials containing homogenized solid material, such as from a stool sample, a tissue sample, or a tissue biopsy, for example. The term “biological sample” also includes materials derived from a tissue culture or a cell culture. Any suitable methods for obtaining a biological sample can be employed; exemplary methods include, e.g., phlebotomy, swab (e.g., buccal swab), and a fine needle aspirate biopsy procedure. Exemplary tissues susceptible to fine needle aspiration include lymph node, lung, thyroid, breast, pancreas, and liver. Samples can also be collected, e.g., by micro dissection (e.g., laser capture micro dissection (LCM) or laser micro dissection (LMD)), bladder wash, smear (e.g., a PAP smear), or ductal lavage. A “biological sample” obtained or derived from a subject includes any such sample that has been processed in any suitable manner after being obtained from the subject. In some embodiments, a biological sample is a plasma sample.

Further, in some embodiments, a biological sample may be derived by taking biological samples from a number of subjects and pooling them, or pooling an aliquot of each subject's biological sample. The pooled sample may be treated as described herein for a sample from a single subject, and, for example, if a poor prognosis is established in the pooled sample, then each subject biological sample can be re-tested to determine which subject(s) have an increased or decreased risk of a CV event.

For purposes of this specification, the phrase “data attributed to a biological sample from a subject” is intended to mean that the data in some form derived from, or were generated using, the biological sample of the subject. The data may have been reformatted, revised, or mathematically altered to some degree after having been generated, such as by conversion from units in one measurement system to units in another measurement system; but, the data are understood to have been derived from, or were generated using, the biological sample.

“Target”, “target molecule”, and “analyte” are used interchangeably herein to refer to any molecule of interest that may be present in a biological sample. A “molecule of interest” includes any minor variation of a particular molecule, such as, in the case of a protein, for example, minor variations in amino acid sequence, disulfide bond formation, glycosylation, lipidation, acetylation, phosphorylation, or any other manipulation or modification, such as conjugation with a labeling component, which does not substantially alter the identity of the molecule. A “target molecule”, “target”, or “analyte” refers to a set of copies of one type or species of molecule or multi-molecular structure. Exemplary target molecules include proteins, polypeptides, nucleic acids, carbohydrates, lipids, polysaccharides, glycoproteins, hormones, receptors, antigens, antibodies, affybodies, antibody mimics, viruses, pathogens, toxic substances, substrates, metabolites, transition state analogs, cofactors, inhibitors, drugs, dyes, nutrients, growth factors, cells, tissues, and any fragment or portion of any of the foregoing. In some embodiments, a target molecule is a protein, in which case the target molecule may be referred to as a “target protein.”

As used herein, a “capture agent’ or “capture reagent” refers to a molecule that is capable of binding specifically to a biomarker. A “target protein capture reagent” refers to a molecule that is capable of binding specifically to a target protein. Nonlimiting exemplary capture reagents include aptamers, antibodies, adnectins, ankyrins, other antibody mimetics and other protein scaffolds, autoantibodies, chimeras, small molecules, nucleic acids, lectins, ligand-binding receptors, imprinted polymers, avimers, peptidomimetics, hormone receptors, cytokine receptors, synthetic receptors, and modifications and fragments of any of the aforementioned capture reagents. In some embodiments, a capture reagent is selected from an aptamer and an antibody.

The term “antibody” refers to full-length antibodies of any species and fragments and derivatives of such antibodies, including Fab fragments, F(ab′)₂ fragments, single chain antibodies, Fv fragments, and single chain Fv fragments. The term “antibody” also refers to synthetically-derived antibodies, such as phage display-derived antibodies and fragments, affybodies, nanobodies, etc.

As used herein, “marker” and “biomarker” are used interchangeably to refer to a target molecule that indicates or is a sign of a normal or abnormal process in a subject or of a disease or other condition in a subject. More specifically, a “marker” or “biomarker” is an anatomic, physiologic, biochemical, or molecular parameter associated with the presence of a specific physiological state or process, whether normal or abnormal, and, if abnormal, whether chronic or acute. Biomarkers are detectable and measurable by a variety of methods including laboratory assays and medical imaging. In some embodiments, a biomarker is a target protein.

As used herein, “biomarker level” and “level” refer to a measurement that is made using any analytical method for detecting the biomarker in a biological sample and that indicates the presence, absence, absolute amount or concentration, relative amount or concentration, titer, a level, an expression level, a ratio of measured levels, or the like, of, for, or corresponding to the biomarker in the biological sample. The exact nature of the “level” depends on the specific design and components of the particular analytical method employed to detect the biomarker.

When a biomarker indicates or is a sign of an abnormal process or a disease or other condition in a subject, that biomarker is generally described as being either over-expressed or under-expressed as compared to an expression level or value of the biomarker that indicates or is a sign of a normal process or an absence of a disease or other condition in a subject. “Up-regulation”, “up-regulated”, “over-expression”, “over-expressed”, and any variations thereof are used interchangeably to refer to a value or level of a biomarker in a biological sample that is greater than a value or level (or range of values or levels) of the biomarker that is typically detected in similar biological samples from healthy or normal subjects. The terms may also refer to a value or level of a biomarker in a biological sample that is greater than a value or level (or range of values or levels) of the biomarker that may be detected at a different stage of a particular disease.

“Down-regulation”, “down-regulated”, “under-expression”, “under-expressed”, and any variations thereof are used interchangeably to refer to a value or level of a biomarker in a biological sample that is less than a value or level (or range of values or levels) of the biomarker that is typically detected in similar biological samples from healthy or normal subjects. The terms may also refer to a value or level of a biomarker in a biological sample that is less than a value or level (or range of values or levels) of the biomarker that may be detected at a different stage of a particular disease.

Further, a biomarker that is either over-expressed or under-expressed can also be referred to as being “differentially expressed” or as having a “differential level” or “differential value” as compared to a “normal” expression level or value of the biomarker that indicates or is a sign of a normal process or an absence of a disease or other condition in a subject. Thus, “differential expression” of a biomarker can also be referred to as a variation from a “normal” expression level of the biomarker.

A “control level” of a target molecule refers to the level of the target molecule in the same sample type from a subject that does not have the disease or condition, or from a subject that is not suspected or at risk of having the disease or condition, or from a subject that has had a primary or first cardiovascular event but not a secondary cardiovascular event, or from a subject that has stable cardiovascular disease. Control level may refer to the average level of the target molecule in samples from a population of subjects that does not have the disease or condition, or that is not suspected or at risk of having the disease or condition, or that has had a primary or first cardiovascular event but not a secondary cardiovascular event, or that has stable cardiovascular disease or a combination thereof.

As used herein, “individual,” “subject,” and “patient” are used interchangeably to refer to a mammal. A mammalian subject can be a human or non-human. In various embodiments, the subject is a human. A healthy or normal subject is a subject in which the disease or condition of interest (including, for example, cardiovascular events such as myocardial infarction, stroke and hospitalization for heart failure) is not detectable by conventional diagnostic methods.

“Diagnose”, “diagnosing”, “diagnosis”, and variations thereof refer to the detection, determination, or recognition of a health status or condition of a subject on the basis of one or more signs, symptoms, data, or other information pertaining to that subject. The health status of a subject can be diagnosed as healthy/normal (i.e., a diagnosis of the absence of a disease or condition) or diagnosed as ill/abnormal (i.e., a diagnosis of the presence, or an assessment of the characteristics, of a disease or condition). The terms “diagnose”, “diagnosing”, “diagnosis”, etc., encompass, with respect to a particular disease or condition, the initial detection of the disease; the characterization or classification of the disease; the detection of the progression, remission, or recurrence of the disease; and the detection of disease response after the administration of a treatment or therapy to the subject. The prediction of risk of a CV event includes distinguishing subjects who have an increased risk of a CV event from subjects who do not.

“Prognose”, “prognosing”, “prognosis”, and variations thereof refer to the prediction of a future course of a disease or condition in a subject who has the disease or condition (e.g., predicting patient survival), and such terms encompass the evaluation of disease or condition response after the administration of a treatment or therapy to the subject.

“Evaluate”, “evaluating”, “evaluation”, and variations thereof encompass both “diagnose” and “prognose” and also encompass determinations or predictions about the future course of a disease or condition in a subject who does not have the disease as well as determinations or predictions regarding the risk that a disease or condition will recur in a subject who apparently has been cured of the disease or has had the condition resolved. The term “evaluate” also encompasses assessing a subject's response to a therapy, such as, for example, predicting whether a subject is likely to respond favorably to a therapeutic agent or is unlikely to respond to a therapeutic agent (or will experience toxic or other undesirable side effects, for example), selecting a therapeutic agent for administration to a subject, or monitoring or determining a subject's response to a therapy that has been administered to the subject. Thus, “evaluating” risk of a CV event can include, for example, any of the following: predicting the future risk of a CV event in a subject; predicting the risk of a CV event in a subject who apparently has no CV issues; predicting a particular type of CV event; predicting the time to a CV event; or determining or predicting a subject's response to a CV treatment or selecting a CV treatment to administer to a subject based upon a determination of the biomarker values derived from the subject's biological sample. Evaluation of risk of a CV event can include embodiments such as the assessment of risk of a CV event on a continuous scale, or classification of risk of a CV event in escalating classifications. Classification of risk includes, for example, classification into two or more classifications such as “Medium Risk of a CV Event;” “High Risk of a CV Event;” and/or “Low Risk of CV Event.” In some embodiments, the evaluation of risk of a CV event is for a defined period. Nonlimiting exemplary defined periods include 1 year, 2 years, 3 years, 4 years, 5 years and more than 5 years.

As used herein, “additional biomedical information” refers to one or more evaluations of a subject, other than using any of the biomarkers described herein, that are associated with CV risk or, more specifically, CV event risk. “Additional biomedical information” includes any of the following: physical descriptors of a subject, including the height and/or weight of a subject; the age of a subject; the gender of a subject; change in weight; the ethnicity of a subject; occupational history; family history of cardiovascular disease (or other circulatory system disorders); the presence of a genetic marker(s) correlating with a higher risk of cardiovascular disease (or other circulatory system disorders) in the subject or a family member alterations in the carotid intima thickness; clinical symptoms such as chest pain, weight gain or loss gene expression values; physical descriptors of a subject, including physical descriptors observed by radiologic imaging; smoking status; alcohol use history; occupational history; dietary habits—salt, saturated fat and cholesterol intake; caffeine consumption; and imaging information such as electrocardiogram, echocardiography, carotid ultrasound for intima-media thickness, flow mediated dilation, pulse wave velocity, ankle-brachial index, stress echocardiography, myocardial perfusion imaging, coronary calcium by CT, high resolution CT angiography, MRI imaging, and other imaging modalities; and the subject's medications. Testing of biomarker levels in combination with an evaluation of any additional biomedical information, including other laboratory tests (e.g., HDL, LDL testing, CRP levels, Nt-proBNP testing, BNP testing, high sensitivity troponin testing, galectin-3 testing, serum albumin testing, creatine testing), may, for example, improve sensitivity, specificity, and/or AUC for prediction of CV events as compared to biomarker testing alone or evaluating any particular item of additional biomedical information alone (e.g., carotid intima thickness imaging alone). Additional biomedical information can be obtained from a subject using routine techniques known in the art, such as from the subject themselves by use of a routine patient questionnaire or health history questionnaire, etc., or from a medical practitioner, etc. Testing of biomarker levels in combination with an evaluation of any additional biomedical information may, for example, improve sensitivity, specificity, and/or thresholds for prediction of CV events (or other cardiovascular-related uses) as compared to biomarker testing alone or evaluating any particular item of additional biomedical information alone (e.g., CT imaging alone).

As used herein, “detecting” or “determining” with respect to a biomarker value includes the use of both the instrument used to observe and record a signal corresponding to a biomarker level and the material/s required to generate that signal. In various embodiments, the biomarker level is detected using any suitable method, including fluorescence, chemiluminescence, surface plasmon resonance, surface acoustic waves, mass spectrometry, infrared spectroscopy, Raman spectroscopy, atomic force microscopy, scanning tunneling microscopy, electrochemical detection methods, nuclear magnetic resonance, quantum dots, and the like.

As used herein, a PCE risk classification is determined according to Goff et al., “2013 ACC/AHA Guideline on the Assessment of Cardiovascular Risk: A Report of the American College of Cardiology/American Heart Association Task Force on Practice Guidelines,” published online in Circulation on Nov. 12, 2013 (Print ISSN: 0009-7322, Online ISSN: 1524-4539). As used herein, a “high” PCE risk classification is a 20.0% or greater predicted 10-year risk for a hard atherosclerotic/cardiovascular disease (ASCVD) event (defined as first occurrence of nonfatal myocardial infarction or coronary heart disease (CHD) death, or fatal or nonfatal stroke); an “intermediate” PCE risk classification is a 10.0-19.9% predicted 10-year risk for a hard ASCVD event; and a “low” PCE risk classification is a <10.0% predicted 10-year risk for a hard ASCVD event. See Goff at page 16, Table 5.

“Solid support” refers herein to any substrate having a surface to which molecules may be attached, directly or indirectly, through either covalent or non-covalent bonds. A “solid support” can have a variety of physical formats, which can include, for example, a membrane; a chip (e.g., a protein chip); a slide (e.g., a glass slide or coverslip); a column; a hollow, solid, semi-solid, pore- or cavity-containing particle, such as, for example, a bead; a gel; a fiber, including a fiber optic material; a matrix; and a sample receptacle. Exemplary sample receptacles include sample wells, tubes, capillaries, vials, and any other vessel, groove or indentation capable of holding a sample. A sample receptacle can be contained on a multi-sample platform, such as a microtiter plate, slide, microfluidics device, and the like. A support can be composed of a natural or synthetic material, an organic or inorganic material. The composition of the solid support on which capture reagents are attached generally depends on the method of attachment (e.g., covalent attachment). Other exemplary receptacles include microdroplets and microfluidic controlled or bulk oil/aqueous emulsions within which assays and related manipulations can occur. Suitable solid supports include, for example, plastics, resins, polysaccharides, silica or silica-based materials, functionalized glass, modified silicon, carbon, metals, inorganic glasses, membranes, nylon, natural fibers (such as, for example, silk, wool and cotton), polymers, and the like. The material composing the solid support can include reactive groups such as, for example, carboxy, amino, or hydroxyl groups, which are used for attachment of the capture reagents. Polymeric solid supports can include, e.g., polystyrene, polyethylene glycol tetraphthalate, polyvinyl acetate, polyvinyl chloride, polyvinyl pyrrolidone, polyacrylonitrile, polymethyl methacrylate, polytetrafluoroethylene, butyl rubber, styrenebutadiene rubber, natural rubber, polyethylene, polypropylene, (poly)tetrafluoroethylene, (poly)vinylidenefluoride, polycarbonate, and polymethylpentene. Suitable solid support particles that can be used include, e.g., encoded particles, such as Luminex®-type encoded particles, magnetic particles, and glass particles.

### Exemplary Uses of Biomarkers

In various exemplary embodiments, methods are provided for evaluating risk or likelihood of a CV event in a subject by detecting one or more biomarker values corresponding to one or more biomarkers that are present in the circulation of a subject, such as in blood, serum or plasma, by any number of analytical methods, including any of the analytical methods described herein. These biomarkers are, for example, differentially expressed in subjects with increased risk of a CV event as compared to subjects without increased risk of a CV event. Detection of the differential expression of a biomarker in a subject can be used, for example, to permit the prediction of risk of a CV event within a 1 year, 2 year, 3 year, 4 year, or 5 year time frame.

In addition to testing biomarker levels as a stand-alone diagnostic test, biomarker levels can also be done in conjunction with determination of single nucleotide polymorphisms (SNPs) or other genetic lesions or variability that are indicative of increased risk of susceptibility of disease or condition. (See, e.g., Amos et al., Nature Genetics 40, 616-622 (2009)). Biomarker levels can also be used in conjunction with radiologic screening. Biomarker levels can also be used in conjunction with relevant symptoms or genetic testing. Detection of any of the biomarkers described herein may be useful after the risk of CV event has been evaluated to guide appropriate clinical care of the subject, including increasing to more aggressive levels of care in high risk subjects after the CV event risk has been determined. In addition to testing biomarker levels in conjunction with relevant symptoms or risk factors, information regarding the biomarkers can also be evaluated in conjunction with other types of data, particularly data that indicates a subject's risk for cardiovascular events (e.g., patient clinical history, symptoms, family history of cardiovascular disease, history of smoking or alcohol use, risk factors such as the presence of a genetic marker(s), and/or status of other biomarkers, etc.). These various data can be assessed by automated methods, such as a computer program/software, which can be embodied in a computer or other apparatus/device.

In addition to testing biomarker levels in conjunction with radiologic screening in high risk subjects (e.g., assessing biomarker levels in conjunction with blockage detected in a coronary angiogram), information regarding the biomarkers can also be evaluated in conjunction with other types of data, particularly data that indicates a subject's risk for having a CV event (e.g., patient clinical history, symptoms, family history of cardiovascular disease, risk factors such as whether or not the subject is a smoker, heavy alcohol user and/or status of other biomarkers, etc.). These various data can be assessed by automated methods, such as a computer program/software, which can be embodied in a computer or other apparatus/device.

Testing of biomarkers can also be associated with guidelines and cardiovascular risk algorithms currently in use in clinical practice. For example, the Framingham Risk Score uses risk factors to provide a risk score, such risk factors including LDL-cholesterol and HDL-cholesterol levels, impaired glucose levels, smoking, systolic blood pressure, and diabetes. The frequency of high-risk patients increases with age, and men comprise a greater proportion of high-risk patients than women.

Any of the described biomarkers may also be used in imaging tests. For example, an imaging agent can be coupled to any of the described biomarkers, which can be used to aid in prediction of risk of a Cardiovascular event, to monitor response to therapeutic interventions, to select for target populations in a clinical trial among other uses.

### Detection and Determination of Biomarkers and Biomarker Levels

A biomarker level for the biomarkers described herein can be detected using any of a variety of known analytical methods. In one embodiment, a biomarker value is detected using a capture reagent. In various embodiments, the capture reagent can be exposed to the biomarker in solution or can be exposed to the biomarker while the capture reagent is immobilized on a solid support. In other embodiments, the capture reagent contains a feature that is reactive with a secondary feature on a solid support. In these embodiments, the capture reagent can be exposed to the biomarker in solution, and then the feature on the capture reagent can be used in conjunction with the secondary feature on the solid support to immobilize the biomarker on the solid support. The capture reagent is selected based on the type of analysis to be conducted. Capture reagents include but are not limited to aptamers, antibodies, adnectins, ankyrins, other antibody mimetics and other protein scaffolds, autoantibodies, chimeras, small molecules, F(ab′)₂ fragments, single chain antibody fragments, Fv fragments, single chain Fv fragments, nucleic acids, lectins, ligand-binding receptors, affybodies, nanobodies, imprinted polymers, avimers, peptidomimetics, hormone receptors, cytokine receptors, and synthetic receptors, and modifications and fragments of these.

In some embodiments, a biomarker level is detected using a biomarker/capture reagent complex.

In some embodiments, the biomarker level is derived from the biomarker/capture reagent complex and is detected indirectly, such as, for example, as a result of a reaction that is subsequent to the biomarker/capture reagent interaction, but is dependent on the formation of the biomarker/capture reagent complex.

In some embodiments, the biomarker level is detected directly from the biomarker in a biological sample.

In some embodiments, biomarkers are detected using a multiplexed format that allows for the simultaneous detection of two or more biomarkers in a biological sample. In some embodiments of the multiplexed format, capture reagents are immobilized, directly or indirectly, covalently or non-covalently, in discrete locations on a solid support. In some embodiments, a multiplexed format uses discrete solid supports where each solid support has a unique capture reagent associated with that solid support, such as, for example quantum dots. In some embodiments, an individual device is used for the detection of each one of multiple biomarkers to be detected in a biological sample. Individual devices can be configured to permit each biomarker in the biological sample to be processed simultaneously. For example, a microtiter plate can be used such that each well in the plate is used to uniquely analyze one or more biomarkers to be detected in a biological sample.

In one or more of the foregoing embodiments, a fluorescent tag can be used to label a component of the biomarker/capture reagent complex to enable the detection of the biomarker level. In various embodiments, the fluorescent label can be conjugated to a capture reagent specific to any of the biomarkers described herein using known techniques, and the fluorescent label can then be used to detect the corresponding biomarker level. Suitable fluorescent labels include rare earth chelates, fluorescein and its derivatives, rhodamine and its derivatives, dansyl, allophycocyanin, PBXL-3, Qdot 605, Lissamine, phycoerythrin, Texas Red, and other such compounds.

In some embodiments, the fluorescent label is a fluorescent dye molecule. In some embodiments, the fluorescent dye molecule includes at least one substituted indolium ring system in which the substituent on the 3-carbon of the indolium ring contains a chemically reactive group or a conjugated substance. In some embodiments, the dye molecule includes an AlexFluor molecule, such as, for example, AlexaFluor 488, AlexaFluor 532, AlexaFluor 647, AlexaFluor 680, or AlexaFluor 700. In other embodiments, the dye molecule includes a first type and a second type of dye molecule, such as, e.g., two different AlexaFluor molecules. In some embodiments, the dye molecule includes a first type and a second type of dye molecule, and the two dye molecules have different emission spectra.

Fluorescence can be measured with a variety of instrumentation compatible with a wide range of assay formats. For example, spectrofluorimeters have been designed to analyze microtiter plates, microscope slides, printed arrays, cuvettes, etc. See Principles of Fluorescence Spectroscopy, by J. R. Lakowicz, Springer Science+Business Media, Inc., 2004. See Bioluminescence & Chemiluminescence: Progress & Current Applications; Philip E. Stanley and Larry J. Kricka editors, World Scientific Publishing Company, January 2002.

In one or more embodiments, a chemiluminescence tag can optionally be used to label a component of the biomarker/capture complex to enable the detection of a biomarker level. Suitable chemiluminescent materials include any of oxalyl chloride, Rodamin 6G, Ru(bipy)₃²⁺, TMAE (tetrakis(dimethylamino)ethylene), Pyrogallol (1,2,3-trihydroxibenzene), Lucigenin, peroxyoxalates, Aryl oxalates, Acridinium esters, dioxetanes, and others.

In some embodiments, the detection method includes an enzyme/substrate combination that generates a detectable signal that corresponds to the biomarker level. Generally, the enzyme catalyzes a chemical alteration of the chromogenic substrate which can be measured using various techniques, including spectrophotometry, fluorescence, and chemiluminescence. Suitable enzymes include, for example, luciferases, luciferin, malate dehydrogenase, urease, horseradish peroxidase (HRPO), alkaline phosphatase, beta-galactosidase, glucoamylase, lysozyme, glucose oxidase, galactose oxidase, and glucose-6-phosphate dehydrogenase, uricase, xanthine oxidase, lactoperoxidase, microperoxidase, and the like.

In some embodiments, the detection method can be a combination of fluorescence, chemiluminescence, radionuclide or enzyme/substrate combinations that generate a measurable signal. In some embodiments, multimodal signaling could have unique and advantageous characteristics in biomarker assay formats.

In some embodiments, the biomarker levels for the biomarkers described herein can be detected using any analytical methods including, singleplex aptamer assays, multiplexed aptamer assays, singleplex or multiplexed immunoassays, mRNA expression profiling, miRNA expression profiling, mass spectrometric analysis, histological/cytological methods, etc. as discussed below.

### Determination of Biomarker Levels Using Aptamer-Based Assays

Assays directed to the detection and quantification of physiologically significant molecules in biological samples and other samples are important tools in scientific research and in the health care field. One class of such assays involves the use of a microarray that includes one or more aptamers immobilized on a solid support. The aptamers are each capable of binding to a target molecule in a highly specific manner and with very high affinity. See, e.g., U.S. Pat. No. 5,475,096 entitled “Nucleic Acid Ligands”; see also, e.g., U.S. Pat. Nos. 6,242,246, 6,458,543, and 6,503,715, each of which is entitled “Nucleic Acid Ligand Diagnostic Biochip”. Once the microarray is contacted with a sample, the aptamers bind to their respective target molecules present in the sample and thereby enable a determination of a biomarker level corresponding to a biomarker.

As used herein, an “aptamer” refers to a nucleic acid that has a specific binding affinity for a target molecule. It is recognized that affinity interactions are a matter of degree; however, in this context, the “specific binding affinity” of an aptamer for its target means that the aptamer binds to its target generally with a much higher degree of affinity than it binds to other components in a test sample. An “aptamer” is a set of copies of one type or species of nucleic acid molecule that comprises a particular nucleotide sequence. An aptamer can include any suitable number of nucleotides, including any number of chemically modified nucleotides. “Aptamers” refers to more than one such set of molecules. Different aptamers can have either the same or different numbers of nucleotides. Aptamers can be DNA or RNA or chemically modified nucleic acids and can be single-stranded, double-stranded, or contain double-stranded regions, and can include higher ordered structures. An aptamer can also be a photoaptamer, where a photoreactive or chemically reactive functional group is included in the aptamer to allow it to be covalently linked to its corresponding target. Any of the aptamer methods disclosed herein can include the use of two or more aptamers that specifically bind the same target molecule. As further described below, an aptamer may include a tag. If an aptamer includes a tag, all copies of the aptamer need not have the same tag. Moreover, if different aptamers each include a tag, these different aptamers can have either the same tag or a different tag.

An aptamer can be identified using any known method, including the SELEX process. Once identified, an aptamer can be prepared or synthesized in accordance with any known method, including chemical synthetic methods and enzymatic synthetic methods.

The terms “SELEX” and “SELEX process” are used interchangeably herein to refer generally to a combination of (1) the selection of aptamers that interact with a target molecule in a desirable manner, for example binding with high affinity to a protein, with (2) the amplification of those selected nucleic acids. The SELEX process can be used to identify aptamers with high affinity to a specific target or biomarker.

SELEX generally includes preparing a candidate mixture of nucleic acids, binding of the candidate mixture to the desired target molecule to form an affinity complex, separating the affinity complexes from the unbound candidate nucleic acids, separating and isolating the nucleic acid from the affinity complex, purifying the nucleic acid, and identifying a specific aptamer sequence. The process may include multiple rounds to further refine the affinity of the selected aptamer. The process can include amplification steps at one or more points in the process. See, e.g., U.S. Pat. No. 5,475,096, entitled “Nucleic Acid Ligands”. The SELEX process can be used to generate an aptamer that covalently binds its target as well as an aptamer that non-covalently binds its target. See, e.g., U.S. Pat. No. 5,705,337 entitled “Systematic Evolution of Nucleic Acid Ligands by Exponential Enrichment: Chemi-SELEX.”

The SELEX process can be used to identify high-affinity aptamers containing modified nucleotides that confer improved characteristics on the aptamer, such as, for example, improved in vivo stability or improved delivery characteristics. Examples of such modifications include chemical substitutions at the ribose and/or phosphate and/or base positions. SELEX process-identified aptamers containing modified nucleotides are described in U.S. Pat. No. 5,660,985, entitled “High Affinity Nucleic Acid Ligands Containing Modified Nucleotides”, which describes oligonucleotides containing nucleotide derivatives chemically modified at the 5′- and 2′-positions of pyrimidines. U.S. Pat. No. 5,580,737, see supra, describes highly specific aptamers containing one or more nucleotides modified with 2′-amino (2′-NH2), 2′-fluoro (2′-F), and/or 2′-O-methyl (2′-OMe). See also, U.S. Patent Application Publication 20090098549, entitled “SELEX and PHOTOSELEX”, which describes nucleic acid libraries having expanded physical and chemical properties and their use in SELEX and photoSELEX.

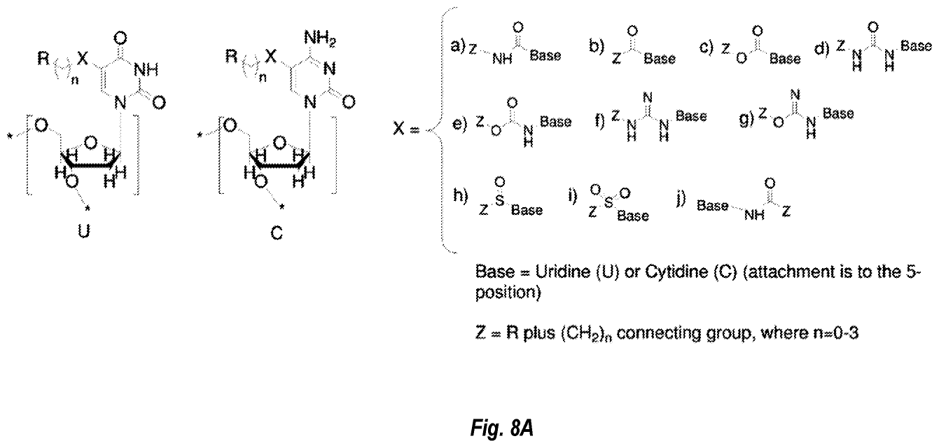

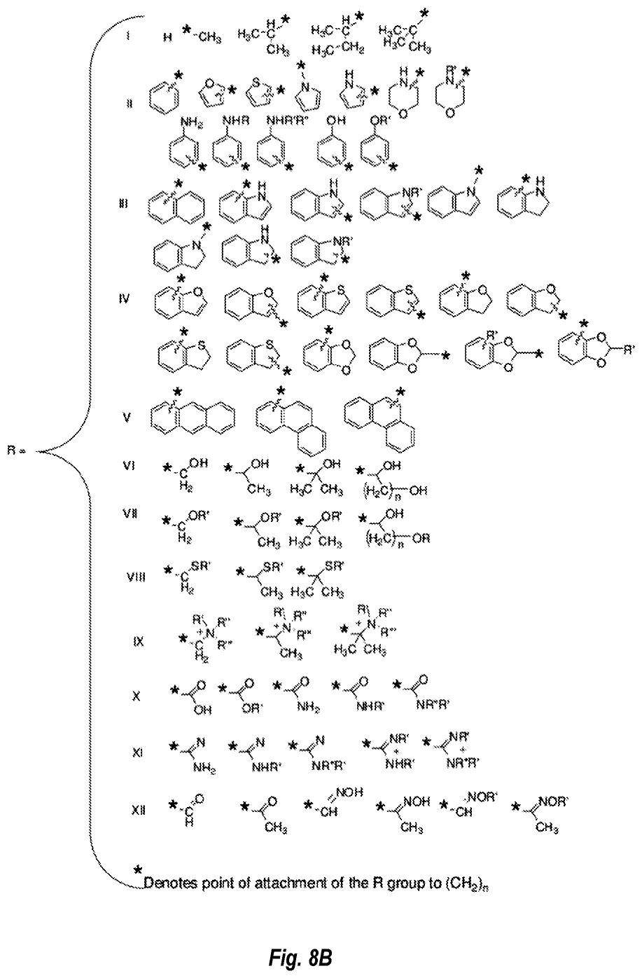

SELEX can also be used to identify aptamers that have desirable off-rate characteristics. See U.S. Publication No. 20090004667, entitled “Method for Generating Aptamers with Improved Off-Rates”, which describes improved SELEX methods for generating aptamers that can bind to target molecules. Methods for producing aptamers and photoaptamers having slower rates of dissociation from their respective target molecules are described. The methods involve contacting the candidate mixture with the target molecule, allowing the formation of nucleic acid-target complexes to occur, and performing a slow off-rate enrichment process wherein nucleic acid-target complexes with fast dissociation rates will dissociate and not reform, while complexes with slow dissociation rates will remain intact. Additionally, the methods include the use of modified nucleotides in the production of candidate nucleic acid mixtures to generate aptamers with improved off-rate performance. Nonlimiting exemplary modified nucleotides include, for example, the modified pyrimidines shown in FIG. 8. In some embodiments, an aptamer comprises at least one nucleotide with a modification, such as a base modification. In some embodiments, an aptamer comprises at least one nucleotide with a hydrophobic modification, such as a hydrophobic base modification, allowing for hydrophobic contacts with a target protein. Such hydrophobic contacts, in some embodiments, contribute to greater affinity and/or slower off-rate binding by the aptamer. Nonlimiting exemplary nucleotides with hydrophobic modifications are shown in FIG. 8. In some embodiments, an aptamer comprises at least two, at least three, at least four, at least five, at least six, at least seven, at least eight, at least nine, or at least 10 nucleotides with hydrophobic modifications, where each hydrophobic modification may be the same or different from the others. In some embodiments, at least one, at least two, at least three, at least four, at least five, at least six, at least seven, at least eight, at least nine, or at least 10 hydrophobic modifications in an aptamer may be independently selected from the hydrophobic modifications shown in FIG. 8.

In some embodiments, a slow off-rate aptamer (including an aptamers comprising at least one nucleotide with a hydrophobic modification) has an off-rate (t½) of ≥30 minutes, ≥60 minutes, ≥90 minutes, ≥120 minutes, ≥150 minutes, ≥180 minutes, ≥210 minutes, or ≥240 minutes.

In some embodiments, an assay employs aptamers that include photoreactive functional groups that enable the aptamers to covalently bind or “photocrosslink” their target molecules. See, e.g., U.S. Pat. No. 6,544,776 entitled “Nucleic Acid Ligand Diagnostic Biochip”. These photoreactive aptamers are also referred to as photoaptamers. See, e.g., U.S. Pat. Nos. 5,763,177, 6,001,577, and 6,291,184, each of which is entitled “Systematic Evolution of Nucleic Acid Ligands by Exponential Enrichment: Photoselection of Nucleic Acid Ligands and Solution SELEX”; see also, e.g., U.S. Pat. No. 6,458,539, entitled “Photoselection of Nucleic Acid Ligands”. After the microarray is contacted with the sample and the photoaptamers have had an opportunity to bind to their target molecules, the photoaptamers are photoactivated, and the solid support is washed to remove any non-specifically bound molecules. Harsh wash conditions may be used, since target molecules that are bound to the photoaptamers are generally not removed, due to the covalent bonds created by the photoactivated functional group(s) on the photoaptamers. In this manner, the assay enables the detection of a biomarker level corresponding to a biomarker in the test sample.

In some assay formats, the aptamers are immobilized on the solid support prior to being contacted with the sample. Under certain circumstances, however, immobilization of the aptamers prior to contact with the sample may not provide an optimal assay. For example, pre-immobilization of the aptamers may result in inefficient mixing of the aptamers with the target molecules on the surface of the solid support, perhaps leading to lengthy reaction times and, therefore, extended incubation periods to permit efficient binding of the aptamers to their target molecules. Further, when photoaptamers are employed in the assay and depending upon the material utilized as a solid support, the solid support may tend to scatter or absorb the light used to effect the formation of covalent bonds between the photoaptamers and their target molecules. Moreover, depending upon the method employed, detection of target molecules bound to their aptamers can be subject to imprecision, since the surface of the solid support may also be exposed to and affected by any labeling agents that are used. Finally, immobilization of the aptamers on the solid support generally involves an aptamer-preparation step (i.e., the immobilization) prior to exposure of the aptamers to the sample, and this preparation step may affect the activity or functionality of the aptamers.

Aptamer assays that permit an aptamer to capture its target in solution and then employ separation steps that are designed to remove specific components of the aptamer-target mixture prior to detection have also been described (see U.S. Publication No. 20090042206, entitled “Multiplexed Analyses of Test Samples”). The described aptamer assay methods enable the detection and quantification of a non-nucleic acid target (e.g., a protein target) in a test sample by detecting and quantifying a nucleic acid (i.e., an aptamer). The described methods create a nucleic acid surrogate (i.e, the aptamer) for detecting and quantifying a non-nucleic acid target, thus allowing the wide variety of nucleic acid technologies, including amplification, to be applied to a broader range of desired targets, including protein targets.

Aptamers can be constructed to facilitate the separation of the assay components from an aptamer biomarker complex (or photoaptamer biomarker covalent complex) and permit isolation of the aptamer for detection and/or quantification. In one embodiment, these constructs can include a cleavable or releasable element within the aptamer sequence. In other embodiments, additional functionality can be introduced into the aptamer, for example, a labeled or detectable component, a spacer component, or a specific binding tag or immobilization element. For example, the aptamer can include a tag connected to the aptamer via a cleavable moiety, a label, a spacer component separating the label, and the cleavable moiety. In one embodiment, a cleavable element is a photocleavable linker. The photocleavable linker can be attached to a biotin moiety and a spacer section, can include an NHS group for derivatization of amines, and can be used to introduce a biotin group to an aptamer, thereby allowing for the release of the aptamer later in an assay method.

Homogenous assays, done with all assay components in solution, do not require separation of sample and reagents prior to the detection of signal. These methods are rapid and easy to use. These methods generate signal based on a molecular capture or binding reagent that reacts with its specific target. In some embodiments of the methods described herein, the molecular capture reagents comprise one or more aptamer or antibody or the like and the specific target of each of the one or more aptamer or antibody of the like may be a biomarker shown in Table 1 or in Table 2.

In some embodiments, a method for signal generation takes advantage of anisotropy signal change due to the interaction of a fluorophore-labeled capture reagent with its specific biomarker target. When the labeled capture reacts with its target, the increased molecular weight causes the rotational motion of the fluorophore attached to the complex to become much slower changing the anisotropy value. By monitoring the anisotropy change, binding events may be used to quantitatively measure the biomarkers in solutions. Other methods include fluorescence polarization assays, molecular beacon methods, time resolved fluorescence quenching, chemiluminescence, fluorescence resonance energy transfer, and the like.

An exemplary solution-based aptamer assay that can be used to detect a biomarker level in a biological sample includes the following: (a) preparing a mixture by contacting the biological sample with an aptamer that includes a first tag and has a specific affinity for the biomarker, wherein an aptamer affinity complex is formed when the biomarker is present in the sample; (b) exposing the mixture to a first solid support including a first capture element, and allowing the first tag to associate with the first capture element; (c) removing any components of the mixture not associated with the first solid support; (d) attaching a second tag to the biomarker component of the aptamer affinity complex; (e) releasing the aptamer affinity complex from the first solid support; (f) exposing the released aptamer affinity complex to a second solid support that includes a second capture element and allowing the second tag to associate with the second capture element; (g) removing any non-complexed aptamer from the mixture by partitioning the non-complexed aptamer from the aptamer affinity complex; (h) eluting the aptamer from the solid support; and (i) detecting the biomarker by detecting the aptamer component of the aptamer affinity complex.

Any means known in the art can be used to detect a biomarker value by detecting the aptamer component of an aptamer affinity complex. A number of different detection methods can be used to detect the aptamer component of an affinity complex, such as, for example, hybridization assays, mass spectroscopy, or QPCR. In some embodiments, nucleic acid sequencing methods can be used to detect the aptamer component of an aptamer affinity complex and thereby detect a biomarker value. Briefly, a test sample can be subjected to any kind of nucleic acid sequencing method to identify and quantify the sequence or sequences of one or more aptamers present in the test sample. In some embodiments, the sequence includes the entire aptamer molecule or any portion of the molecule that may be used to uniquely identify the molecule. In other embodiments, the identifying sequencing is a specific sequence added to the aptamer; such sequences are often referred to as “tags,” “barcodes,” or “zipcodes.” In some embodiments, the sequencing method includes enzymatic steps to amplify the aptamer sequence or to convert any kind of nucleic acid, including RNA and DNA that contain chemical modifications to any position, to any other kind of nucleic acid appropriate for sequencing.

In some embodiments, the sequencing method includes one or more cloning steps. In other embodiments the sequencing method includes a direct sequencing method without cloning.

In some embodiments, the sequencing method includes a directed approach with specific primers that target one or more aptamers in the test sample. In other embodiments, the sequencing method includes a shotgun approach that targets all aptamers in the test sample.

In some embodiments, the sequencing method includes enzymatic steps to amplify the molecule targeted for sequencing. In other embodiments, the sequencing method directly sequences single molecules. An exemplary nucleic acid sequencing-based method that can be used to detect a biomarker value corresponding to a biomarker in a biological sample includes the following: (a) converting a mixture of aptamers that contain chemically modified nucleotides to unmodified nucleic acids with an enzymatic step; (b) shotgun sequencing the resulting unmodified nucleic acids with a massively parallel sequencing platform such as, for example, the 454 Sequencing System (454 Life Sciences/Roche), the Illumina Sequencing System (Illumina), the ABI SOLiD Sequencing System (Applied Biosystems), the HeliScope Single Molecule Sequencer (Helicos Biosciences), or the Pacific Biosciences Real Time Single-Molecule Sequencing System (Pacific BioSciences) or the Polonator G Sequencing System (Dover Systems); and (c) identifying and quantifying the aptamers present in the mixture by specific sequence and sequence count.

A nonlimiting exemplary method of detecting biomarkers in a biological sample using aptamers is described in Example 1. See also Kraemer et al., 2011, PLoS One 6(10): e26332.

### Determination of Biomarker Levels Using Immunoassays

Immunoassay methods are based on the reaction of an antibody to its corresponding target or analyte and can detect the analyte in a sample depending on the specific assay format. To improve specificity and sensitivity of an assay method based on immuno-reactivity, monoclonal antibodies and fragments thereof are often used because of their specific epitope recognition. Polyclonal antibodies have also been successfully used in various immunoassays because of their increased affinity for the target as compared to monoclonal antibodies. Immunoassays have been designed for use with a wide range of biological sample matrices. Immunoassay formats have been designed to provide qualitative, semi-quantitative, and quantitative results.

Quantitative results are generated through the use of a standard curve created with known concentrations of the specific analyte to be detected. The response or signal from an unknown sample is plotted onto the standard curve, and a quantity or level corresponding to the target in the unknown sample is established.

Numerous immunoassay formats have been designed. ELISA or EIA can be quantitative for the detection of an analyte. This method relies on attachment of a label to either the analyte or the antibody and the label component includes, either directly or indirectly, an enzyme. ELISA tests may be formatted for direct, indirect, competitive, or sandwich detection of the analyte. Other methods rely on labels such as, for example, radioisotopes (I¹²⁵) or fluorescence. Additional techniques include, for example, agglutination, nephelometry, turbidimetry, Western blot, immunoprecipitation, immunocytochemistry, immunohistochemistry, flow cytometry, Luminex assay, and others (see ImmunoAssay: A Practical Guide, edited by Brian Law, published by Taylor & Francis, Ltd., 2005 edition).

Exemplary assay formats include enzyme-linked immunosorbent assay (ELISA), radioimmunoassay, fluorescent, chemiluminescence, and fluorescence resonance energy transfer (FRET) or time resolved-FRET (TR-FRET) immunoassays. Examples of procedures for detecting biomarkers include biomarker immunoprecipitation followed by quantitative methods that allow size and peptide level discrimination, such as gel electrophoresis, capillary electrophoresis, planar electrochromatography, and the like.

Methods of detecting and/or for quantifying a detectable label or signal generating material depend on the nature of the label. The products of reactions catalyzed by appropriate enzymes (where the detectable label is an enzyme; see above) can be, without limitation, fluorescent, luminescent, or radioactive or they may absorb visible or ultraviolet light. Examples of detectors suitable for detecting such detectable labels include, without limitation, x-ray film, radioactivity counters, scintillation counters, spectrophotometers, colorimeters, fluorometers, luminometers, and densitometers.

Any of the methods for detection can be performed in any format that allows for any suitable preparation, processing, and analysis of the reactions. This can be, for example, in multi-well assay plates (e.g., 96 wells or 386 wells) or using any suitable array or microarray. Stock solutions for various agents can be made manually or robotically, and all subsequent pipetting, diluting, mixing, distribution, washing, incubating, sample readout, data collection and analysis can be done robotically using commercially available analysis software, robotics, and detection instrumentation capable of detecting a detectable label.

### Determination of Biomarker Levels Using Gene Expression Profiling

Measuring mRNA in a biological sample may, in some embodiments, be used as a surrogate for detection of the level of the corresponding protein in the biological sample. Thus, in some embodiments, a biomarker or biomarker panel described herein can be detected by detecting the appropriate RNA.

In some embodiments, mRNA expression levels are measured by reverse transcription quantitative polymerase chain reaction (RT-PCR followed with qPCR). RT-PCR is used to create a cDNA from the mRNA. The cDNA may be used in a qPCR assay to produce fluorescence as the DNA amplification process progresses. By comparison to a standard curve, qPCR can produce an absolute measurement such as number of copies of mRNA per cell. Northern blots, microarrays, Invader assays, and RT-PCR combined with capillary electrophoresis have all been used to measure expression levels of mRNA in a sample. See Gene Expression Profiling: Methods and Protocols, Richard A. Shimkets, editor, Humana Press, 2004.

### Detection of Biomarkers Using In Vivo Molecular Imaging Technologies

In some embodiments, a biomarker described herein may be used in molecular imaging tests. For example, an imaging agent can be coupled to a capture reagent, which can be used to detect the biomarker in vivo.

In vivo imaging technologies provide non-invasive methods for determining the state of a particular disease in the body of a subject. For example, entire portions of the body, or even the entire body, may be viewed as a three dimensional image, thereby providing valuable information concerning morphology and structures in the body. Such technologies may be combined with the detection of the biomarkers described herein to provide information concerning the biomarker in vivo.

The use of in vivo molecular imaging technologies is expanding due to various advances in technology. These advances include the development of new contrast agents or labels, such as radiolabels and/or fluorescent labels, which can provide strong signals within the body; and the development of powerful new imaging technology, which can detect and analyze these signals from outside the body, with sufficient sensitivity and accuracy to provide useful information. The contrast agent can be visualized in an appropriate imaging system, thereby providing an image of the portion or portions of the body in which the contrast agent is located. The contrast agent may be bound to or associated with a capture reagent, such as an aptamer or an antibody, for example, and/or with a peptide or protein, or an oligonucleotide (for example, for the detection of gene expression), or a complex containing any of these with one or more macromolecules and/or other particulate forms.

The contrast agent may also feature a radioactive atom that is useful in imaging. Suitable radioactive atoms include technetium-99m or iodine-123 for scintigraphic studies. Other readily detectable moieties include, for example, spin labels for magnetic resonance imaging (MRI) such as, for example, iodine-123 again, iodine-131, indium-111, fluorine-19, carbon-13, nitrogen-15, oxygen-17, gadolinium, manganese or iron. Such labels are well known in the art and could easily be selected by one of ordinary skill in the art.

Standard imaging techniques include but are not limited to magnetic resonance imaging, computed tomography scanning, positron emission tomography (PET), single photon emission computed tomography (SPECT), and the like. For diagnostic in vivo imaging, the type of detection instrument available is a major factor in selecting a given contrast agent, such as a given radionuclide and the particular biomarker that it is used to target (protein, mRNA, and the like). The radionuclide chosen typically has a type of decay that is detectable by a given type of instrument. Also, when selecting a radionuclide for in vivo diagnosis, its half-life should be long enough to enable detection at the time of maximum uptake by the target tissue but short enough that deleterious radiation of the host is minimized.

Exemplary imaging techniques include but are not limited to PET and SPECT, which are imaging techniques in which a radionuclide is synthetically or locally administered to a subject. The subsequent uptake of the radiotracer is measured over time and used to obtain information about the targeted tissue and the biomarker. Because of the high-energy (gamma-ray) emissions of the specific isotopes employed and the sensitivity and sophistication of the instruments used to detect them, the two-dimensional distribution of radioactivity may be inferred from outside of the body.

Commonly used positron-emitting nuclides in PET include, for example, carbon-11, nitrogen-13, oxygen-15, and fluorine-18. Isotopes that decay by electron capture and/or gamma-emission are used in SPECT and include, for example iodine-123 and technetium-99m. An exemplary method for labeling amino acids with technetium-99m is the reduction of pertechnetate ion in the presence of a chelating precursor to form the labile technetium-99m-precursor complex, which, in turn, reacts with the metal binding group of a bifunctionally modified chemotactic peptide to form a technetium-99m-chemotactic peptide conjugate.

Antibodies are frequently used for such in vivo imaging diagnostic methods. The preparation and use of antibodies for in vivo diagnosis is well known in the art. Similarly, aptamers may be used for such in vivo imaging diagnostic methods. For example, an aptamer that was used to identify a particular biomarker described herein may be appropriately labeled and injected into a subject to detect the biomarker in vivo. The label used will be selected in accordance with the imaging modality to be used, as previously described. Aptamer-directed imaging agents could have unique and advantageous characteristics relating to tissue penetration, tissue distribution, kinetics, elimination, potency, and selectivity as compared to other imaging agents.

Such techniques may also optionally be performed with labeled oligonucleotides, for example, for detection of gene expression through imaging with antisense oligonucleotides. These methods are used for in situ hybridization, for example, with fluorescent molecules or radionuclides as the label. Other methods for detection of gene expression include, for example, detection of the activity of a reporter gene.

Another general type of imaging technology is optical imaging, in which fluorescent signals within the subject are detected by an optical device that is external to the subject. These signals may be due to actual fluorescence and/or to bioluminescence. Improvements in the sensitivity of optical detection devices have increased the usefulness of optical imaging for in vivo diagnostic assays.

For a review of other techniques, see N. Blow, Nature Methods, 6, 465-469, 2009.

### Determination of Biomarker Levels Using Mass Spectrometry Methods

A variety of configurations of mass spectrometers can be used to detect biomarker levels. Several types of mass spectrometers are available or can be produced with various configurations. In general, a mass spectrometer has the following major components: a sample inlet, an ion source, a mass analyzer, a detector, a vacuum system, and instrument-control system, and a data system. Difference in the sample inlet, ion source, and mass analyzer generally define the type of instrument and its capabilities. For example, an inlet can be a capillary-column liquid chromatography source or can be a direct probe or stage such as used in matrix-assisted laser desorption. Common ion sources are, for example, electrospray, including nanospray and microspray or matrix-assisted laser desorption. Common mass analyzers include a quadrupole mass filter, ion trap mass analyzer and time-of-flight mass analyzer. Additional mass spectrometry methods are well known in the art (see Burlingame et al. Anal. Chem. 70:647 R-716R (1998); Kinter and Sherman, New York (2000)).

Protein biomarkers and biomarker levels can be detected and measured by any of the following: electrospray ionization mass spectrometry (ESI-MS), ESI-MS/MS, ESI-MS/(MS)n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), tandem time-of-flight (TOF/TOF) technology, called ultraflex III TOF/TOF, atmospheric pressure chemical ionization mass spectrometry (APCI-MS), APCI-MS/MS, APCI-(MS)N, atmospheric pressure photoionization mass spectrometry (APPI-MS), APPI-MS/MS, and APPI-(MS)N, quadrupole mass spectrometry, Fourier transform mass spectrometry (FTMS), quantitative mass spectrometry, and ion trap mass spectrometry.

Sample preparation strategies are used to label and enrich samples before mass spectroscopic characterization of protein biomarkers and determination biomarker levels. Labeling methods include but are not limited to isobaric tag for relative and absolute quantitation (iTRAQ) and stable isotope labeling with amino acids in cell culture (SILAC). Capture reagents used to selectively enrich samples for candidate biomarker proteins prior to mass spectroscopic analysis include but are not limited to aptamers, antibodies, nucleic acid probes, chimeras, small molecules, an F(ab′)₂ fragment, a single chain antibody fragment, an Fv fragment, a single chain Fv fragment, a nucleic acid, a lectin, a ligand-binding receptor, affybodies, nanobodies, ankyrins, domain antibodies, alternative antibody scaffolds (e.g. diabodies etc) imprinted polymers, avimers, peptidomimetics, peptoids, peptide nucleic acids, threose nucleic acid, a hormone receptor, a cytokine receptor, and synthetic receptors, and modifications and fragments of these.

### Determination of Biomarker Levels Using a Proximity Ligation Assay

A proximity ligation assay can be used to determine biomarker values. Briefly, a test sample is contacted with a pair of affinity probes that may be a pair of antibodies or a pair of aptamers, with each member of the pair extended with an oligonucleotide. The targets for the pair of affinity probes may be two distinct determinates on one protein or one determinate on each of two different proteins, which may exist as homo- or hetero-multimeric complexes. When probes bind to the target determinates, the free ends of the oligonucleotide extensions are brought into sufficiently close proximity to hybridize together. The hybridization of the oligonucleotide extensions is facilitated by a common connector oligonucleotide which serves to bridge together the oligonucleotide extensions when they are positioned in sufficient proximity. Once the oligonucleotide extensions of the probes are hybridized, the ends of the extensions are joined together by enzymatic DNA ligation.

Each oligonucleotide extension comprises a primer site for PCR amplification. Once the oligonucleotide extensions are ligated together, the oligonucleotides form a continuous DNA sequence which, through PCR amplification, reveals information regarding the identity and amount of the target protein, as well as, information regarding protein-protein interactions where the target determinates are on two different proteins. Proximity ligation can provide a highly sensitive and specific assay for real-time protein concentration and interaction information through use of real-time PCR. Probes that do not bind the determinates of interest do not have the corresponding oligonucleotide extensions brought into proximity and no ligation or PCR amplification can proceed, resulting in no signal being produced.

The foregoing assays enable the detection of biomarker values that are useful in methods for prediction of risk of CV events, where the methods comprise detecting, in a biological sample from a subject, at least five, at least six, at least seven, at least eight, at least nine, at least ten, at least eleven, at least twelve, or thirteen biomarkers selected from the biomarkers in Table 1; or at least eight, at least nine, at least ten, at least eleven, at least twelve, at least thirteen, at least fourteen, at least fifteen, at least sixteen, at least seventeen, at least eighteen, at least nineteen, at least twenty, at least twenty-one, at least twenty-two, at least twenty-three, at least twenty-four, at least twenty-five, at least twenty-six, or all twenty-seven of the biomarkers in Table 2; wherein a classification, as described below, using the biomarker values indicates whether the subject has elevated risk of a CV event occurring within a 1 year, 2 year, 3 year, or 4 year time period. In accordance with any of the methods described herein, biomarker values can be detected and classified individually or they can be detected and classified collectively, as for example in a multiplex assay format.

### Classification of Biomarkers and Calculation of Disease Scores

In some embodiments, a biomarker “signature” for a given diagnostic test contains a set of biomarkers, each biomarker having characteristic levels in the populations of interest. Characteristic levels, in some embodiments, may refer to the mean or average of the biomarker levels for the subjects in a particular group. In some embodiments, a diagnostic method described herein can be used to assign an unknown sample from a subject into one of two groups, either at increased risk of a CV event or not.

The assignment of a sample into one of two or more groups is known as classification, and the procedure used to accomplish this assignment is known as a classifier or a classification method. Classification methods may also be referred to as scoring methods. There are many classification methods that can be used to construct a diagnostic classifier from a set of biomarker levels. In some instances, classification methods are performed using supervised learning techniques in which a data set is collected using samples obtained from subjects within two (or more, for multiple classification states) distinct groups one wishes to distinguish. Since the class (group or population) to which each sample belongs is known in advance for each sample, the classification method can be trained to give the desired classification response. It is also possible to use unsupervised learning techniques to produce a diagnostic classifier.

Common approaches for developing diagnostic classifiers include decision trees; bagging+boosting+forests; rule inference based learning; Parzen Windows; linear models; logistic; neural network methods; unsupervised clustering; K-means; hierarchical ascending/descending; semi-supervised learning; prototype methods; nearest neighbor; kernel density estimation; support vector machines; hidden Markov models; Boltzmann Learning; and classifiers may be combined either simply or in ways which minimize particular objective functions. For a review, see, e.g., Pattern Classification, R. O. Duda, et al., editors, John Wiley & Sons, 2nd edition, 2001; see also, The Elements of Statistical Learning—Data Mining, Inference, and Prediction, T. Hastie, et al., editors, Springer Science+Business Media, LLC, 2nd edition, 2009.

To produce a classifier using supervised learning techniques, a set of samples called training data are obtained. In the context of diagnostic tests, training data includes samples from the distinct groups (classes) to which unknown samples will later be assigned. For example, samples collected from subjects in a control population and subjects in a particular disease population can constitute training data to develop a classifier that can classify unknown samples (or, more particularly, the subjects from whom the samples were obtained) as either having the disease or being free from the disease. The development of the classifier from the training data is known as training the classifier. Specific details on classifier training depend on the nature of the supervised learning technique. Training a naïve Bayesian classifier is an example of such a supervised learning technique (see, e.g., Pattern Classification, R. O. Duda, et al., editors, John Wiley & Sons, 2nd edition, 2001; see also, The Elements of Statistical Learning—Data Mining, Inference, and Prediction, T. Hastie, et al., editors, Springer Science+Business Media, LLC, 2nd edition, 2009). Training of a naïve Bayesian classifier is described, e.g., in U.S. Publication Nos: 2012/0101002 and 2012/0077695.

Since typically there are many more potential biomarker levels than samples in a training set, care must be used to avoid over-fitting. Over-fitting occurs when a statistical model describes random error or noise instead of the underlying relationship. Over-fitting can be avoided in a variety of way, including, for example, by limiting the number of biomarkers used in developing the classifier, by assuming that the biomarker responses are independent of one another, by limiting the complexity of the underlying statistical model employed, and by ensuring that the underlying statistical model conforms to the data.

An illustrative example of the development of a diagnostic test using a set of biomarkers includes the application of a naïve Bayes classifier, a simple probabilistic classifier based on Bayes theorem with strict independent treatment of the biomarkers. Each biomarker is described by a class-dependent probability density function (pdf) for the measured RFU values or log RFU (relative fluorescence units) values in each class. The joint pdfs for the set of biomarkers in one class is assumed to be the product of the individual class-dependent pdfs for each biomarker. Training a naïve Bayes classifier in this context amounts to assigning parameters (“parameterization”) to characterize the class dependent pdfs. Any underlying model for the class-dependent pdfs may be used, but the model should generally conform to the data observed in the training set.

The performance of the naïve Bayes classifier is dependent upon the number and quality of the biomarkers used to construct and train the classifier. A single biomarker will perform in accordance with its KS-distance (Kolmogorov-Smirnov). The addition of subsequent biomarkers with good KS distances (>0.3, for example) will, in general, improve the classification performance if the subsequently added biomarkers are independent of the first biomarker. Using the sensitivity plus specificity as a classifier score, many high scoring classifiers can be generated with a variation of a greedy algorithm. (A greedy algorithm is any algorithm that follows the problem solving metaheuristic of making the locally optimal choice at each stage with the hope of finding the global optimum.)

Another way to depict classifier performance is through a receiver operating characteristic (ROC), or simply ROC curve or ROC plot. The ROC is a graphical plot of the sensitivity, or true positive rate, vs. false positive rate (1-specificity or 1-true negative rate), for a binary classifier system as its discrimination threshold is varied. The ROC can also be represented equivalently by plotting the fraction of true positives out of the positives (TPR=true positive rate) vs. the fraction of false positives out of the negatives (FPR=false positive rate). Also known as a Relative Operating Characteristic curve, because it is a comparison of two operating characteristics (TPR & FPR) as the criterion changes. The area under the ROC curve (AUC) is commonly used as a summary measure of diagnostic accuracy. It can take values from 0.0 to 1.0. The AUC has an important statistical property: the AUC of a classifier is equivalent to the probability that the classifier will rank a randomly chosen positive instance higher than a randomly chosen negative instance (Fawcett T, 2006. An introduction to ROC analysis. Pattern Recognition Letters. 27: 861-874). This is equivalent to the Wilcoxon test of ranks (Hanley, J. A., McNeil, B. J., 1982. The meaning and use of the area under a receiver operating characteristic (ROC) curve. Radiology 143, 29-36). Another way of describing performance of a diagnostic test in relation to a known reference standard is the net reclassification index: the ability of the new test to correctly upgrade or downgrade risk when compared with the reference standard test. See, e.g., Pencina et al., 2011, Stat. Med. 30: 11-21. While the AUC under the ROC curve is optimal for assessing performance of a 2-class classifier, stratified and personalized medicine relies upon the inference that the population contains more classes than 2. For such comparisons the hazard ratio of the upper vs. lower quartiles (or other stratifications such as deciles) can be used more appropriately.

The risk and likelihood predictions enabled herein may be applied to subjects in primary care or in specialist cardiovascular centers, or even direct to the consumer. In some embodiments, the classifiers used to predict events may involve some calibration to the population to which they are applied—for example there may be variations due to ethnicity or geography. Such calibrations, in some embodiments, may be established in advance from large population studies, so when applied to an individual patient these are incorporated prior to making a risk prediction. A venous blood sample is taken, processed appropriately and analyzed as described herein. Once the analysis is complete, the risk predictions may be made mathematically, with or without incorporating other metadata from medical records described herein such as genetic or demographic. Various forms of output of information are possible depending on the level of expertise of the consumer. For consumers seeking the simplest type of output the information may be, in some embodiments, “is this person likely to have an event in the next x years (where x is 1-4), yes/no” or alternatively akin to a “traffic light” red/orange/green or its verbal or written equivalent such as high/medium/low risk. For consumers seeking greater detail, in some embodiments, the risk may be output as a number or a graphic illustrating the probability of an event per unit time as a continuous score, or a greater number of strata (such as deciles), and/or the average time to event and/or the most likely type of event. In some embodiments, the output may include therapeutic recommendations. Longitudinal monitoring of the same patient over time will enable graphics showing response to interventions or lifestyle changes. In some embodiments, more than one type of output may be provided at the same time to fulfill the needs of the patient and of individual members of the care team with differing levels of expertise.

In some embodiments, the biomarkers shown in Table 1 or Table 2 are detected in a blood sample (such as a plasma sample or a serum sample) from a subject, for example, using aptamers, such as slow off-rate aptamers. The log RFU values are used to calculate a subject's risk or likelihood of having a CV event, or a prognostic index (PI).

Given the PI, the probability that the subject will suffer a cardiovascular event (CV event) in the next “t” years is given by the formula:

$$P r [ T \leq t ] = 1 - {e}^{- e ( \frac{Log ( t ) - PI}{s} )} ,$$

where PI is the prognostic index (or linear predictor) and s is the associated scale parameter for the extreme value distribution. In various embodiments, “t” is 5 years or less, 4 years or less, 3 years or less, or 2 years or less.

### Kits

Any combination of the biomarkers described herein can be detected using a suitable kit, such as for use in performing the methods disclosed herein. Furthermore, any kit can contain one or more detectable labels as described herein, such as a fluorescent moiety, etc.

In some embodiments, a kit includes (a) one or more capture reagents (such as, for example, at least one aptamer or antibody) for detecting one or more biomarkers in a biological sample, wherein the biomarkers include at least five, at least six, at least seven, at least eight, at least nine, at least ten, at least eleven, at least twelve, or thirteen biomarkers selected from the biomarkers in Table 1; or at least eight, at least nine, at least ten, at least eleven, at least twelve, at least thirteen, at least fourteen, at least fifteen, at least sixteen, at least seventeen, at least eighteen, at least nineteen, at least twenty, at least twenty-one, at least twenty-two, at least twenty-three, at least twenty-four, at least twenty-five, at least twenty-six, or all twenty-seven of the biomarkers in Table 2; and optionally (b) one or more software or computer program products for classifying the subject from whom the biological sample was obtained as either having or not having increased risk of a CV event or for determining the likelihood that the subject has increased risk of a CV event, as further described herein. Alternatively, rather than one or more computer program products, one or more instructions for manually performing the above steps by a human can be provided.

In some embodiments, a kit comprises a solid support, a capture reagent, and a signal generating material. The kit can also include instructions for using the devices and reagents, handling the sample, and analyzing the data. Further the kit may be used with a computer system or software to analyze and report the result of the analysis of the biological sample.

The kits can also contain one or more reagents (e.g., solubilization buffers, detergents, washes, or buffers) for processing a biological sample. Any of the kits described herein can also include, e.g., buffers, blocking agents, mass spectrometry matrix materials, antibody capture agents, positive control samples, negative control samples, software and information such as protocols, guidance and reference data.

In some embodiments kits are provided for the analysis of CV event risk status, wherein the kits comprise PCR primers for one or more aptamers specific to biomarkers described herein. In some embodiments, a kit may further include instructions for use and correlation of the biomarkers with prediction of risk of a CV event. In some embodiments, a kit may also include a DNA array containing the complement of one or more of the aptamers specific for the biomarkers described herein, reagents, and/or enzymes for amplifying or isolating sample DNA. In some embodiments, kits may include reagents for real-time PCR, for example, TaqMan probes and/or primers, and enzymes.

For example, a kit can comprise (a) reagents comprising at least one capture reagent for determining the level of one or more biomarkers in a test sample, and optionally (b) one or more algorithms or computer programs for performing the steps of comparing the amount of each biomarker quantified in the test sample to one or more predetermined cutoffs. In some embodiments, an algorithm or computer program assigns a score for each biomarker quantified based on said comparison and, in some embodiments, combines the assigned scores for each biomarker quantified to obtain a total score. Further, in some embodiments, an algorithm or computer program compares the total score with a predetermined score, and uses the comparison to determine whether a subject has an increased risk of a CV event. Alternatively, rather than one or more algorithms or computer programs, one or more instructions for manually performing the above steps by a human can be provided.

### Biomarker Panels

In some embodiments, one or more of the biomarkers listed in Table 1 is detected. In some embodiments, all of the biomarkers listed in the table below are detected. In some embodiments, the level of each protein listed in Table 1 is detected. In some embodiments, the detecting of the one or more biomarkers or all of the biomarkers is performed in order to determine the risk or likelihood a subject will have a primary CV event within a defined time period. In some such embodiments, the defined time period is one year, two years, three years, four years, or five years. In some embodiments, the defined time period is four years.

TABLE 1

| Protein | Gene | UniProt ID | Name |
|---|---|---|---|
| N-terminal | NPPB | P16860 | N-terminal pro-BNP |
| pro-BNP | | | |
| MMP-12 | MMP12 | P39900 | Macrophage metalloelastase |
| sTREM-1 | TREM1 | Q9NP99 | Triggering receptor expressed on myeloid |
| | | | cells 1 |
| Antithrombin | SERPINC1 | P01008 | Antithrombin-III |
| III | | | |
| GPR56 | GPR56 | Q9Y653 | Adhesion G protein-coupled receptor G1 |
| Gelsolin | GSN | P06396 | Gelsolin |
| ST4S6 | CHST15 | Q7LFX5 | Carbohydrate Sulfotransferase 15 |
| CHSTC | CHST12 | Q9NRB3 | Carbohydrate Sulfotransferase 12 |
| FSH | FSHB | P01225 | Follicle Stimulating Hormone |
| IL-1 sRII | IL1R2 | P27930 | Interleukin-1 Receptor Type 2 |
| PLXB2 | PLXNB2 | O15031 | Plexin-B2 |
| SAP | APCS | P02743 | Serum Amyloid P-Component |
| TFPI | TFPI | P10646 | Tissue Factor Pathway Inhibitor |

In some embodiments, one or more of the biomarkers listed in Table 2 is detected. In some embodiments, all of the biomarkers listed in the table below are detected. In some embodiments, the level of each protein listed in Table 2 is detected. In some embodiments, the detecting of the one or more biomarkers or all of the biomarkers is performed in order to determine the risk or likelihood a subject will have a secondary CV event within a defined time period. In some such embodiments, the defined time period is one year, two years, three years, four years, or five ears. In some embodiments, the defined time period is four ears.

TABLE 2

| Protein | Gene | UniProt ID | Name |
|---|---|---|---|
| BNP | NPPB | P16860 | Natriuretic peptides B |
| MMP-12 | MMP12 | P39900 | Macrophage metalloelastase |
| sTREM-1 | TREM1 | Q9NP99 | Triggering receptor expressed on myeloid |
| | | | cells 1 |
| SVEP1 | SVEP1 | Q4LDE5 | Sushi; von Willebrand factor type A; EGF |
| | | | and pentraxin domain-containing protein 1 |
| ARL11 | ARL11 | Q969Q4 | ADP-ribosylation factor-like protein 11 |
| ANTR2 | ANTXR2 | P58335 | Anthrax toxin receptor 2 |
| CA125 | MUC16 | Q8WXI7 | Mucin-16 |
| GOLM1 | GOLM1 | Q8NBJ4 | Golgi membrane protein 1 |
| PPR1A | PPP1R1A | Q13522 | Protein phosphatase 1 regulatory subunit |
| | | | 1A |
| ERBB3 | ERBB3 | P21860 | Receptor tyrosine-protein kinase erbB-3 |
| suPAR | PLAUR | Q03405 | Urokinase plasminogen activator surface |
| | | | receptor |
| GDF-11/8 | GDF11 MSTN | O95390 O14793 | Growth/differentiation factor 11/8 |
| JAM-B | JAM2 | P57087 | Junctional adhesion molecule B |
| ATS13 | ADAMTS13 | Q76LX8 | A disintegrin and metalloproteinase with |
| | | | thrombospondin motifs 13 |
| Spondin-1 | SPON1 | Q9HCB6 | Spondin-1 |
| NCAM-120 | NCAM1 | P13591 | Neural cell adhesion molecule 1; 120 kDa |
| | | | isoform |
| TFF3 | TFF3 | Q07654 | Trefoil factor 3 |
| SIRT2 | SIRT2 | Q8IXJ6 | NAD-dependent protein deacetylase |
| | | | sirtuin-2 |
| ANP | NPPA | P01160 | Atrial natriuretic factor |
| NELL1 | NELL1 | Q92832 | Protein kinase C-binding protein NELL1 |
| LRP11 | LRP11 | Q86VZ4 | Low-density lipoprotein receptor-related |
| | | | protein 11 |
| NDST1 | NDST1 | P52848 | Bifunctional heparan sulfate |
| | | | N-deacetylase/N-sulfotransferase 1 |
| PTPRJ | PTPRJ | Q12913 | Receptor-type tyrosine-protein |
| | | | phosphatase eta |
| CILP2 | CILP2 | Q8IUL8 | Cartilage intermediate layer protein 2 |
| CA2D3 | CACNA2D3 | Q8IZS8 | Voltage-dependent calcium channel |
| | | | subunit alpha-2/delta-3 |
| ITI heavy | ITIH2 | P19823 | Inter-alpha-trypsin inhibitor heavy chain |
| chain H2 | | | H2 |
| IGDC4 | IGDCC4 | Q8TDY8 | Immunoglobulin superfamily DCC |
| | | | subclass member 4 |

### Computer Methods and Software

A method for assessing the risk or likelihood of a CV event in a subject can comprise the following: 1) obtain a biological sample; 2) perform an analytical method to detect and measure a biomarker or set of biomarkers in a panel in the biological sample; 3) optionally perform any data normalization or standardization; 4) determine each biomarker level; and 5) report the results. In some embodiments, the results are calibrated to the population/ethnicity of the subject. In some embodiments, the biomarker levels are combined in some way and a single value for the combined biomarker levels is reported. In this approach, in some embodiments, the score may be a single number determined from the integration of all the biomarkers that is compared to a pre-set threshold value that is an indication of the presence or absence of disease. Or the diagnostic or predictive score may be a series of bars that each represent a biomarker value and the pattern of the responses may be compared to a pre-set pattern for determination of the presence or absence of disease, condition or the increased risk (or not) of an event.

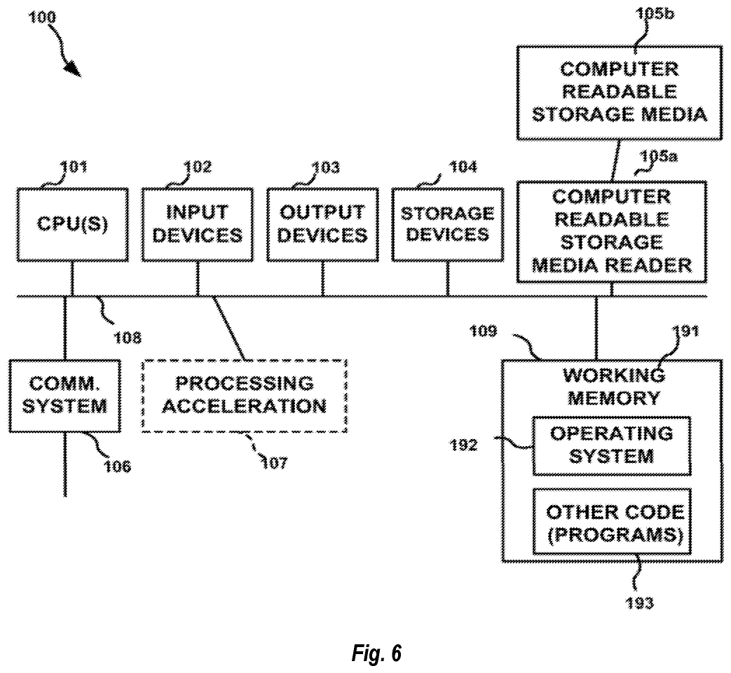

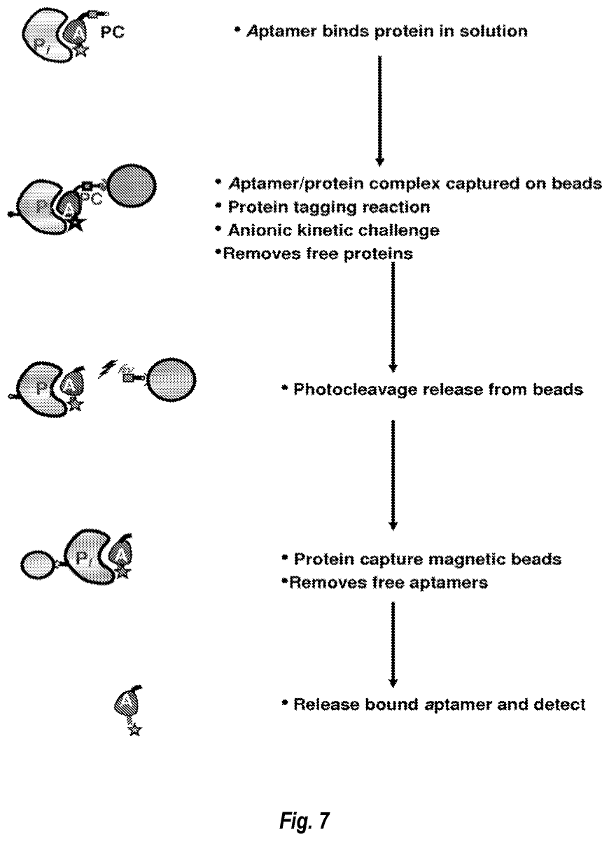

At least some embodiments of the methods described herein can be implemented with the use of a computer. An example of a computer system 100 is shown in FIG. 6. With reference to FIG. 6, system 100 is shown comprised of hardware elements that are electrically coupled via bus 108, including a processor 101, input device 102, output device 103, storage device 104, computer-readable storage media reader 105a, communications system 106, processing acceleration (e.g., DSP or special-purpose processors) 107 and memory 109. Computer-readable storage media reader 105a is further coupled to computer-readable storage media 105b, the combination comprehensively representing remote, local, fixed and/or removable storage devices plus storage media, memory, etc. for temporarily and/or more permanently containing computer-readable information, which can include storage device 104, memory 109 and/or any other such accessible system 100 resource. System 100 also comprises software elements (shown as being currently located within working memory 191) including an operating system 192 and other code 193, such as programs, data and the like.

With respect to FIG. 6, system 100 has extensive flexibility and configurability. Thus, for example, a single architecture might be utilized to implement one or more servers that can be further configured in accordance with currently desirable protocols, protocol variations, extensions, etc. However, it will be apparent to those skilled in the art that embodiments may well be utilized in accordance with more specific application requirements. For example, one or more system elements might be implemented as sub-elements within a system 100 component (e.g., within communications system 106). Customized hardware might also be utilized and/or particular elements might be implemented in hardware, software or both. Further, while connection to other computing devices such as network input/output devices (not shown) may be employed, it is to be understood that wired, wireless, modem, and/or other connection or connections to other computing devices might also be utilized.

In one aspect, the system can comprise a database containing features of biomarkers characteristic of prediction of risk of a CV event. The biomarker data (or biomarker information) can be utilized as an input to the computer for use as part of a computer implemented method. The biomarker data can include the data as described herein.

In one aspect, the system further comprises one or more devices for providing input data to the one or more processors.

The system further comprises a memory for storing a data set of ranked data elements.

In another aspect, the device for providing input data comprises a detector for detecting the characteristic of the data element, e.g., such as a mass spectrometer or gene chip reader.

The system additionally may comprise a database management system. User requests or queries can be formatted in an appropriate language understood by the database management system that processes the query to extract the relevant information from the database of training sets.

The system may be connectable to a network to which a network server and one or more clients are connected. The network may be a local area network (LAN) or a wide area network (WAN), as is known in the art. Preferably, the server includes the hardware necessary for running computer program products (e.g., software) to access database data for processing user requests.

The system may include an operating system (e.g., UNIX or Linux) for executing instructions from a database management system. In one aspect, the operating system can operate on a global communications network, such as the internet, and utilize a global communications network server to connect to such a network.

The system may include one or more devices that comprise a graphical display interface comprising interface elements such as buttons, pull down menus, scroll bars, fields for entering text, and the like as are routinely found in graphical user interfaces known in the art. Requests entered on a user interface can be transmitted to an application program in the system for formatting to search for relevant information in one or more of the system databases. Requests or queries entered by a user may be constructed in any suitable database language.

The graphical user interface may be generated by a graphical user interface code as part of the operating system and can be used to input data and/or to display inputted data. The result of processed data can be displayed in the interface, printed on a printer in communication with the system, saved in a memory device, and/or transmitted over the network or can be provided in the form of the computer readable medium.

The system can be in communication with an input device for providing data regarding data elements to the system (e.g., expression values). In one aspect, the input device can include a gene expression profiling system including, e.g., a mass spectrometer, gene chip or array reader, and the like.

The methods and apparatus for analyzing CV event risk prediction biomarker information according to various embodiments may be implemented in any suitable manner, for example, using a computer program operating on a computer system. A conventional computer system comprising a processor and a random access memory, such as a remotely-accessible application server, network server, personal computer or workstation may be used. Additional computer system components may include memory devices or information storage systems, such as a mass storage system and a user interface, for example a conventional monitor, keyboard and tracking device. The computer system may be a stand-alone system or part of a network of computers including a server and one or more databases.

The CV event risk prediction biomarker analysis system can provide functions and operations to complete data analysis, such as data gathering, processing, analysis, reporting and/or diagnosis. For example, in one embodiment, the computer system can execute the computer program that may receive, store, search, analyze, and report information relating to the CV event risk prediction biomarkers. The computer program may comprise multiple modules performing various functions or operations, such as a processing module for processing raw data and generating supplemental data and an analysis module for analyzing raw data and supplemental data to generate a CV event risk prediction status and/or diagnosis or risk calculation. Calculation of risk status for a CV event may optionally comprise generating or collecting any other information, including additional biomedical information, regarding the condition of the subject relative to the disease, condition or event, identifying whether further tests may be desirable, or otherwise evaluating the health status of the subject.

Some embodiments described herein can be implemented so as to include a computer program product. A computer program product may include a computer readable medium having computer readable program code embodied in the medium for causing an application program to execute on a computer with a database.

As used herein, a “computer program product” refers to an organized set of instructions in the form of natural or programming language statements that are contained on a physical media of any nature (e.g., written, electronic, magnetic, optical or otherwise) and that may be used with a computer or other automated data processing system. Such programming language statements, when executed by a computer or data processing system, cause the computer or data processing system to act in accordance with the particular content of the statements. Computer program products include without limitation: programs in source and object code and/or test or data libraries embedded in a computer readable medium. Furthermore, the computer program product that enables a computer system or data processing equipment device to act in pre-selected ways may be provided in a number of forms, including, but not limited to, original source code, assembly code, object code, machine language, encrypted or compressed versions of the foregoing and any and all equivalents.

In one aspect, a computer program product is provided for evaluation of the risk of a CV event. The computer program product includes a computer readable medium embodying program code executable by a processor of a computing device or system, the program code comprising: code that retrieves data attributed to a biological sample from a subject, wherein the data comprises biomarker levels that each correspond to one of the biomarkers in Table 1 or Table 2; and code that executes a classification method that indicates a CV event risk status of the subject as a function of the biomarker values.

In still another aspect, a computer program product is provided for indicating a likelihood or risk of a CV event. The computer program product includes a computer readable medium embodying program code executable by a processor of a computing device or system, the program code comprising: code that retrieves data attributed to a biological sample from a subject, wherein the data comprises at least one biomarker value corresponding to at least one biomarker in the biological sample selected from the biomarkers provided in Table 1 or Table 2; and code that executes a classification method that indicates a CV event risk status of the subject as a function of the biomarker value.

While various embodiments have been described as methods or apparatuses, it should be understood that embodiments can be implemented through code coupled with a computer, e.g., code resident on a computer or accessible by the computer. For example, software and databases could be utilized to implement many of the methods discussed above. Thus, in addition to embodiments accomplished by hardware, it is also noted that these embodiments can be accomplished through the use of an article of manufacture comprised of a computer usable medium having a computer readable program code embodied therein, which causes the enablement of the functions disclosed in this description. Therefore, it is desired that embodiments also be considered protected by this patent in their program code means as well. Furthermore, the embodiments may be embodied as code stored in a computer-readable memory of virtually any kind including, without limitation, RAM, ROM, magnetic media, optical media, or magneto-optical media. Even more generally, the embodiments could be implemented in software, or in hardware, or any combination thereof including, but not limited to, software running on a general purpose processor, microcode, programmable logic arrays (PLAs), or application-specific integrated circuits (ASICs).

It is also envisioned that embodiments could be accomplished as computer signals embodied in a carrier wave, as well as signals (e.g., electrical and optical) propagated through a transmission medium. Thus, the various types of information discussed above could be formatted in a structure, such as a data structure, and transmitted as an electrical signal through a transmission medium or stored on a computer readable medium.

It is also noted that many of the structures, materials, and acts recited herein can be recited as means for performing a function or step for performing a function. Therefore, it should be understood that such language is entitled to cover all such structures, materials, or acts disclosed within this specification and their equivalents, including the matter incorporated by reference.

The utilization of the biomarkers disclosed herein, and the various methods for determining biomarker values are described in detail above with respect to evaluation of risk of a CV event. However, the application of the process, the use of identified biomarkers, and the methods for determining biomarker values are fully applicable to other specific types of cardiovascular conditions, to any other disease or medical condition, or to the identification of subjects who may or may not be benefited by an ancillary medical treatment.

### Other Methods

In some embodiments, the biomarkers and methods described herein are used to determine a medical insurance premium or coverage decision and/or a life insurance premium or coverage decision. In some embodiments, the results of the methods described herein are used to determine a medical insurance premium and/or a life insurance premium. In some such instances, an organization that provides medical insurance or life insurance requests or otherwise obtains information concerning a subject's risk or likelihood of a CV event and uses that information to determine an appropriate medical insurance or life insurance premium for the subject. In some embodiments, the test is requested by, and paid for by, the organization that provides medical insurance or life insurance. In some embodiments, the test is used by the potential acquirer of a practice or health system or company to predict future liabilities or costs should the acquisition go ahead.

In some embodiments, the biomarkers and methods described herein are used to predict and/or manage the utilization of medical resources. In some such embodiments, the methods are not carried out for the purpose of such prediction, but the information obtained from the method is used in such a prediction and/or management of the utilization of medical resources. For example, a testing facility or hospital may assemble information from the present methods for many subjects in order to predict and/or manage the utilization of medical resources at a particular facility or in a particular geographic area.

### EXAMPLES

The following examples are provided for illustrative purposes only and are not intended to limit the scope of the application as defined by the appended claims. Routine molecular biology techniques described in the following examples can be carried out as described in standard laboratory manuals, such as Sambrook et al., Molecular Cloning: A Laboratory Manual, 3rd. ed., Cold Spring Harbor Laboratory Press, Cold Spring Harbor, N.Y., (2001).

### Example 1: Exemplary Biomarker Detection Using Aptamers

An exemplary method of detecting one or more biomarker proteins in a sample is described, e.g., in Kraemer et al., PLoS One 6(10): e26332, and is described below. Three different methods of quantification: microarray-based hybridization, a Luminex bead-based method, and qPCR, are described.

### Reagents

HEPES, NaCl, KCl, EDTA, EGTA, MgCl₂ and Tween-20 may be purchased, e.g., from Fisher Biosciences. Dextran sulfate sodium salt (DxSO4), nominally 8000 molecular weight, may be purchased, e.g., from AIC and is dialyzed against deionized water for at least 20 hours with one exchange. KOD EX DNA polymerase may be purchased, e.g., from VWR. Tetramethylammonium chloride and CAPSO may be purchased, e.g., from Sigma-Aldrich and streptavidin-phycoerythrin (SAPE) may be purchased, e.g., from Moss Inc. 4-(2-Aminoethyl)-benzenesulfonylfluoride hydrochloride (AEBSF) may be purchased, e.g., from Gold Biotechnology. Streptavidin-coated 96-well plates may be purchased, e.g., from Thermo Scientific (Pierce Streptavidin Coated Plates HBC, clear, 96-well, product number 15500 or 15501). NHS-PEO4-biotin may be purchased, e.g., from Thermo Scientific (EZ-Link NHS-PEO4-Biotin, product number 21329), dissolved in anhydrous DMSO, and may be stored frozen in single-use aliquots. IL-8, MIP-4, Lipocalin-2, RANTES, MMP-7, and MMP-9 may be purchased, e.g., from R&D Systems. Resistin and MCP-1 may be purchased, e.g., from PeproTech, and tPA may be purchased, e.g., from VWR.

### Nucleic Acids

Conventional (including amine- and biotin-substituted) oligodeoxynucleotides may be purchased, e.g., from Integrated DNA Technologies (IDT). Z-Block is a single-stranded oligodeoxynucleotide of sequence 5′-(AC-BnBn)7-AC-3′, where Bn indicates a benzyl-substituted deoxyuridine residue. Z-block may be synthesized using conventional phosphoramidite chemistry. Aptamer capture reagents may also be synthesized by conventional phosphoramidite chemistry, and may be purified, for example, on a 21.5×75 mm PRP-3 column, operating at 80° C. on a Waters Autopurification 2767 system (or Waters 600 series semi-automated system), using, for example, a timberline TL-600 or TL-150 heater and a gradient of triethylammonium bicarbonate (TEAB)/ACN to elute product. Detection is performed at 260 nm and fractions are collected across the main peak prior to pooling best fractions.

### Buffers

Buffer SB18 is composed of 40 mM HEPES, 101 mM NaCl, 5 mM KCl, 5 mM MgCl2, and 0.05% (v/v) Tween 20 adjusted to pH 7.5 with NaOH. Buffer SB17 is SB18 supplemented with 1 mM trisodium EDTA. Buffer PB1 is composed of 10 mM HEPES, 101 mM NaCl, 5 mM KCl, 5 mM MgCl2, 1 mM trisodium EDTA and 0.05% (v/v) Tween-20 adjusted to pH 7.5 with NaOH. CAPSO elution buffer consists of 100 mM CAPSO pH 10.0 and 1 M NaCl. Neutralization buffer contains of 500 mM HEPES, 500 mM HCl, and 0.05% (v/v) Tween-20. Agilent Hybridization Buffer is a proprietary formulation that is supplied as part of a kit (Oligo aCGH/ChIP-on-chip Hybridization Kit). Agilent Wash Buffer 1 is a proprietary formulation (Oligo aCGH/ChIP-on-chip Wash Buffer 1, Agilent). Agilent Wash Buffer 2 is a proprietary formulation (Oligo aCGH/ChIP-on-chip Wash Buffer 2, Agilent). TMAC hybridization solution consists of 4.5 M tetramethylammonium chloride, 6 mM trisodium EDTA, 75 mM Tris-HCl (pH 8.0), and 0.15% (v/v) Sarkosyl. KOD buffer (10-fold concentrated) consists of 1200 mM Tris-HCl, 15 mM MgSO4, 100 mM KCl, 60 mM (NH4)2SO4, 1% v/v Triton-X 100 and 1 mg/mL BSA.

### Sample Preparation

Serum (stored at −80° C. in 100 μL aliquots) is thawed in a 25° C. water bath for 10 minutes, then stored on ice prior to sample dilution. Samples are mixed by gentle vortexing for 8 seconds. A 6% serum sample solution is prepared by dilution into 0.94× SB17 supplemented with 0.6 mM MgCl2, 1 mM trisodium EGTA, 0.8 mM AEBSF, and 2 μM Z-Block. A portion of the 6% serum stock solution is diluted 10-fold in SB17 to create a 0.6% serum stock. 6% and 0.6% stocks are used, in some embodiments, to detect high- and low-abundance analytes, respectively.

### Capture Reagent (Aptamer) and Streptavidin Plate Preparation

Aptamers are grouped into 2 mixes according to the relative abundance of their cognate analytes (or biomarkers). Stock concentrations are 4 nM for each aptamer, and the final concentration of each aptamer is 0.5 nM. Aptamer stock mixes are diluted 4-fold in SB17 buffer, heated to 95° C. for 5 min and cooled to 37° C. over a 15 minute period prior to use. This denaturation-renaturation cycle is intended to normalize aptamer conformer distributions and thus ensure reproducible aptamer activity in spite of variable histories. Streptavidin plates are washed twice with 150 μL buffer PB1 prior to use.

### Incubation and Plate Capture

Heat-cooled 2× Aptamer mixes (55 μL) are combined with an equal volume of 6% or 0.6% serum dilutions, producing mixes containing 3% and 0.3% serum. The plates are sealed with a Silicone Sealing Mat (Axymat Silicone sealing mat, VWR) and incubated for 1.5 h at 37° C. Mixes are then transferred to the wells of a washed 96-well streptavidin plate and further incubated on an Eppendorf Thermomixer set at 37° C., with shaking at 800 rpm, for two hours.

### Manual Assay

Unless otherwise specified, liquid is removed by dumping, followed by two taps onto layered paper towels. Wash volumes are 150 μL and all shaking incubations are done on an Eppendorf Thermomixer set at 25° C., 800 rpm. Mixes are removed by pipetting, and plates are washed twice for 1 minute with buffer PB1 supplemented with 1 mM dextran sulfate and 500 μM biotin, then 4 times for 15 seconds with buffer PB1. A freshly made solution of 1 mM NHS-PEO4-biotin in buffer PB1 (150 μL/well) is added, and plates are incubated for 5 minutes with shaking. The NHS-biotin solution is removed, and plates washed 3 times with buffer PB1 supplemented with 20 mM glycine, and 3 times with buffer PB1. Eighty-five μL of buffer PB1 supplemented with 1 mM DxSO4 is then added to each well, and plates are irradiated under a BlackRay UV lamp (nominal wavelength 365 nm) at a distance of 5 cm for 20 minutes with shaking. Samples are transferred to a fresh, washed streptavidin-coated plate, or an unused well of the existing washed streptavidin plate, combining high and low sample dilution mixtures into a single well. Samples are incubated at room temperature with shaking for 10 minutes. Unadsorbed material is removed and the plates washed 8 times for 15 seconds each with buffer PB1 supplemented with 30% glycerol. Plates are then washed once with buffer PB1. Aptamers are eluted for 5 minutes at room temperature with 100 μL CAPSO elution buffer. 90 μL of the eluate is transferred to a 96-well HybAid plate and 10 μL neutralization buffer is added.

### Semi-Automated Assay

Streptavidin plates bearing adsorbed mixes are placed on the deck of a BioTek EL406 plate washer, which is programmed to perform the following steps: unadsorbed material is removed by aspiration, and wells are washed 4 times with 300 μL of buffer PB1 supplemented with 1 mM dextran sulfate and 500 μM biotin. Wells are then washed 3 times with 300 μL buffer PB1. One hundred fifty μL of a freshly prepared (from a 100 mM stock in DMSO) solution of 1 mM NHS-PEO4-biotin in buffer PB1 is added. Plates are incubated for 5 minutes with shaking. Liquid is aspirated, and wells are washed 8 times with 300 μL buffer PB1 supplemented with 10 mM glycine. One hundred μL of buffer PB1 supplemented with 1 mM dextran sulfate are added. After these automated steps, plates are removed from the plate washer and placed on a thermoshaker mounted under a UV light source (BlackRay, nominal wavelength 365 nm) at a distance of 5 cm for 20 minutes. The thermoshaker is set at 800 rpm and 25° C. After 20 minutes irradiation, samples are manually transferred to a fresh, washed streptavidin plate (or to an unused well of the existing washed plate). High-abundance (3% serum+3% aptamer mix) and low-abundance reaction mixes (0.3% serum+0.3% aptamer mix) are combined into a single well at this point. This “Catch-2” plate is placed on the deck of BioTek EL406 plate washer, which is programmed to perform the following steps: the plate is incubated for 10 minutes with shaking. Liquid is aspirated, and wells are washed 21 times with 300 μL buffer PB1 supplemented with 30% glycerol. Wells are washed 5 times with 300 μL buffer PB1, and the final wash is aspirated. One hundred μL CAPSO elution buffer are added, and aptamers are eluted for 5 minutes with shaking. Following these automated steps, the plate is then removed from the deck of the plate washer, and 90 μL aliquots of the samples are transferred manually to the wells of a HybAid 96-well plate that contains 10 μL neutralization buffer.

### Hybridization to Custom Agilent 8×15 k Microarrays

24 μL of the neutralized eluate is transferred to a new 96-well plate and 6 μL of 10× Agilent Block (Oligo aCGH/ChIP-on-chip Hybridization Kit, Large Volume, Agilent 5188-5380), containing a set of hybridization controls composed of 10 Cy3 aptamers is added to each well. Thirty μL 2× Agilent Hybridization buffer is added to each sample and mixed. Forty μL of the resulting hybridization solution is manually pipetted into each “well” of the hybridization gasket slide (Hybridization Gasket Slide, 8-microarray per slide format, Agilent). Custom Agilent microarray slides, bearing 10 probes per array complementary to 40 nucleotide random region of each aptamer with a 20× dT linker, are placed onto the gasket slides according to the manufacturers' protocol. The assembly (Hybridization Chamber Kit—SureHyb-enabled, Agilent) is clamped and incubated for 19 hours at 60° C. while rotating at 20 rpm.

### Post Hybridization Washing

Approximately 400 mL Agilent Wash Buffer 1 is placed into each of two separate glass staining dishes. Slides (no more than two at a time) are disassembled and separated while submerged in Wash Buffer 1, then transferred to a slide rack in a second staining dish also containing Wash Buffer 1. Slides are incubated for an additional 5 minutes in Wash Buffer 1 with stirring. Slides are transferred to Wash Buffer 2 pre-equilibrated to 37° C. and incubated for 5 minutes with stirring. Slides are transferred to a fourth staining dish containing acetonitrile, and incubated for 5 minutes with stirring.

### Microarray Imaging

Microarray slides are imaged with an Agilent G2565CA Microarray Scanner System, using the Cy3-channel at 5 μm resolution at 100% PMT setting, and the XRD option enabled at 0.05. The resulting TIFF images are processed using Agilent feature extraction software version 10.5.1.1 with the GE1_105_Dec08 protocol.

### Luminex Probe Design

Probes immobilized to beads have 40 deoxynucleotides complementary to the 3′ end of the 40 nucleotide random region of the target aptamer. The aptamer complementary region is coupled to Luminex Microspheres through a hexaethyleneglycol (HEG) linker bearing a 5′ amino terminus. Biotinylated detection deoxyoligonucleotides comprise 17-21 deoxynucleotides complementary to the 5′ primer region of target aptamers. Biotin moieties are appended to the 3′ ends of detection oligos.

### Coupling of Probes to Luminex Microspheres

Probes are coupled to Luminex Microplex Microspheres essentially per the manufacturer's instructions, but with the following modifications: amino-terminal oligonucleotide amounts are 0.08 nMol per 2.5×10⁶ microspheres, and the second EDC addition is 5 μL at 10 mg/mL. Coupling reactions are performed in an Eppendorf ThermoShaker set at 25° C. and 600 rpm.

### Microsphere Hybridization

Microsphere stock solutions (about 40000 microspheres/μL) are vortexed and sonicated in a Health Sonics ultrasonic cleaner (Model: T1.9C) for 60 seconds to suspend the microspheres. Suspended microspheres are diluted to 2000 microspheres per reaction in 1.5× TMAC hybridization solutions and mixed by vortexing and sonication. Thirty-three μL per reaction of the bead mixture are transferred into a 96-well HybAid plate. Seven μL of 15 nM biotinylated detection oligonucleotide stock in 1× TE buffer are added to each reaction and mixed. Ten μL of neutralized assay sample are added and the plate is sealed with a silicon cap mat seal. The plate is first incubated at 96° C. for 5 minutes and incubated at 50° C. without agitation overnight in a conventional hybridization oven. A filter plate (Dura pore, Millipore part number MSBVN1250, 1.2 μm pore size) is prewetted with 75 μL 1×TMAC hybridization solution supplemented with 0.5% (w/v) BSA. The entire sample volume from the hybridization reaction is transferred to the filter plate. The hybridization plate is rinsed with 75 μL 1×TMAC hybridization solution containing 0.5% BSA and any remaining material is transferred to the filter plate. Samples are filtered under slow vacuum, with 150 μL buffer evacuated over about 8 seconds. The filter plate is washed once with 75 μL 1×TMAC hybridization solution containing 0.5% BSA and the microspheres in the filter plate are resuspended in 75 μL 1×TMAC hybridization solution containing 0.5% BSA. The filter plate is protected from light and incubated on an Eppendorf Thermalmixer R for 5 minutes at 1000 rpm. The filter plate is then washed once with 75 μL 1× TMAC hybridization solution containing 0.5% BSA. 75 μL of 10 μg/mL streptavidin phycoerythrin (SAPE-100, MOSS, Inc.) in 1×TMAC hybridization solution is added to each reaction and incubated on Eppendorf Thermalmixer R at 25° C. at 1000 rpm for 60 minutes. The filter plate is washed twice with 75 μL 1×TMAC hybridization solution containing 0.5% BSA and the microspheres in the filter plate are resuspended in 75 μL 1×TMAC hybridization solution containing 0.5% BSA. The filter plate is then incubated protected from light on an Eppendorf Thermalmixer R for 5 minutes, 1000 rpm. The filter plate is then washed once with 75 μL 1× TMAC hybridization solution containing 0.5% BSA. Microspheres are resuspended in 75 μL 1× TMAC hybridization solution supplemented with 0.5% BSA, and analyzed on a Luminex 100 instrument running XPonent 3.0 software. At least 100 microspheres are counted per bead type, under high PMT calibration and a doublet discriminator setting of 7500 to 18000.

### OPCR Read-Out

Standard curves for qPCR are prepared in water ranging from 108 to 102 copies with 10-fold dilutions and a no-template control. Neutralized assay samples are diluted 40-fold into diH2O. The qPCR master mix is prepared at 2× final concentration (2×KOD buffer, 400 μM dNTP mix, 400 nM forward and reverse primer mix, 2×SYBR Green I and 0.5 U KOD EX). Ten μL of 2× qPCR master mix is added to 10 μL of diluted assay sample. qPCR is run on a BioRad MyIQ iCycler with 2 minutes at 96° C. followed by 40 cycles of 96° C. for 5 seconds and 72° C. for 30 seconds.

### Example 2. Cardiovascular Event Model for Prediction of Primary Cardiovascular Events

In order to predict the risk or likelihood that a subject with no known history of cardiovascular disease will have a primary CV event within four years, a cardiovascular disease (CVD) primary model containing a panel of 13 biomarker proteins was developed. The primary CV event was defined as myocardial infarction, stroke, trans-ischemic attack, hospitalization for heart failure, or death attributed to CVD. The training/verification analysis was developed using the HUNT3 data set, which is a case-cohort study design enriched for CVD primary events. The study includes 2,515 people, 41.51% of whom had a CVD event within five years. See Krokstad et al., Int J Epidemiol. 2013; 42:968-977. The data were split into 80% for training and 20% for verification. Predictions in an independent replication set from the Whitehall II study (See Marmot et al., “Health inequalities among British Civil Servants: the Whitehall II study.” Lancet 1991; 337: 1387-1393) was conducted during the validation phase.

### Model

The cardiovascular disease (CVD) primary model is an accelerated failure time (AFT) parametric survival model with a Weibull distribution. This model has 13 biomarkers, age and interactions with age as its features. A 4-year risk was reported. The predictions were binned into four risk bins and three risk categories. Scoring and corresponding actual event rates in the training set over 4 years are reported in Table 3.

TABLE 3

| | | Minimum | Maximum | Observed | Proportion |
|---|---|---|---|---|---|
| Risk | | Risk | Risk | Event | to |
| Category | Risk Bin | Percentage | Percentage | Rate | Report |
| Low Risk | 1x | 0 | ≤2.15 | 1.9% | 2 in 100 |
| Medium | 2x-3x | >2.15 | ≤5.05 | 3.2% | 3 in 100 |
| Risk | 4x-5x | >5.05 | ≤7.7 | 5.3% | 5 in 100 |
| High Risk | 6x and | >7.7 | ≤100 | 10% | 10 in 100 |
| | above | | | | |

### Results

The concordance index (C-index), area under the ROC curve (AUC), and the category-free net reclassification index (NRI) relative to the refit PCE model (refit to the HUNT3 training data) and the published PCE model for 5-year predictions are given in Table 4 below. The final model was also evaluated on the 20% holdout HUNT3 verification dataset.

TABLE 4

| | | | | NRI |
|---|---|---|---|---|
| Model | Dataset | AUC | C-index | (NRI+/NRI−) |
| CVD Primary | Training data | 0.7 | 0.7 | 0.218 |
| Model | | | | (0.127/0.091) |
| | Verification data | 0.64 | 0.64 | 0.009 |
| | | | | (0.005/0.004) |
| Refit PCE Model | Training data | 0.67 | 0.67 | −0.067 |
| | | | | (−0.039/−0.028) |
| | Verification data | 0.65 | 0.65 | −0.28 |
| | | | | (−0.16/−0.12) |

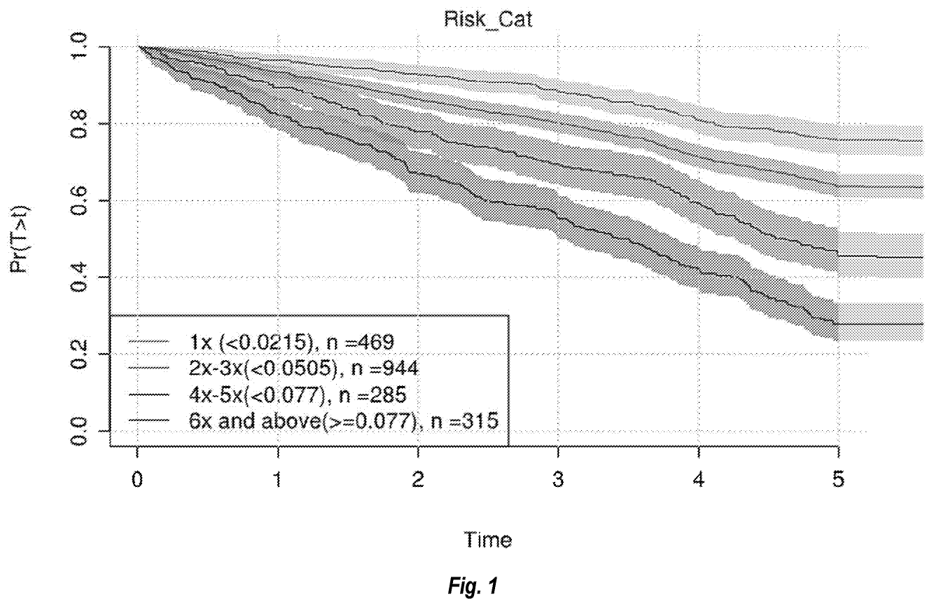

In the HUNT3 training set, 468 patients had a PCE risk score below 7.5%. The closest calculated proteomic risk cutoff at 4 years was 2.15%, with 469 patients having a predicted risk lower than 2.15%. Thus, the healthy baseline stratum was defined as individuals with predicted proteomic risk at 4 years<2.15%. The mean proteomic risk in this population was 1.53%. Four risk bins were calculated as 1×, 2×-3×, 4×-5×, and 6× and above (see Table 3). FIG. 1 depicts Kaplan-Meier survival curves, stratified by the four risk bins, for the HUNT3 training set. FIG. 1 provides an overview of how the empirical distribution of CVD primary events over time separates between the different predicted risk bin groups, with shaded regions representing 95% confidence intervals of the Kaplan-Meier estimates. FIG. 1 shows the separation between the risk bins clearly, with non-overlapping survival distributions at 4 years.

The final model was also evaluated on all individuals (all PCE risk scores including <0.05) in the training and verification datasets. The results are shown in Table 5. The final model performed better than the competing refit clinical PCE model on all individuals as well.

TABLE 5

| | | | | NRI |
|---|---|---|---|---|
| Model | Dataset | AUC | C-index | (NRI+/NRI−) |
| Final CVD | Training data | 0.69 | 0.69 | 0.217 |
| Primary Model | | | | (0.127/0.091) |
| | Verification data | 0.64 | 0.64 | −0.036 |
| | | | | (−0.019/−0.017) |
| Refit PCE Model | Training data | 0.65 | 0.65 | −0.094 |
| | | | | (−0.055/−0.039) |
| | Verification data | 0.64 | 0.65 | −0.33 |
| | | | | (−0.19/−0.14) |

The CVD primary model was characterized further in refinement for several parameters. No significant effects were seen based on sex or in a preliminary interfering substances assessment. The model was applied to 2005 replicates of the assay QC sample and the predictions were reproducible, with a mean of 0.05 and a standard deviation of 0.008. No major variations were seen based on sample processing time.

### Validation

Model validation was performed on the Whitehall II dataset with 265 individuals, 101 of whom had a CV event (38.11%). The analyte RFU values in the dataset were log 10 transformed prior to analysis. Out-of-range log 10 RFU values were imputed using the aptamer-specific maximum and minimum values calculated via winsorization during model development using HUNT3 training data. The final proteomic model was then evaluated on the dataset at the 5-year timepoint, and Net Reclassification Index (NRI) was calculated as a comparison to the published PCE model, shown in Table 6.

TABLE 6

| Metric | Value |
|---|---|
| NRI (NRI+/NRI−) | 0.176 (0.120/0.055) |
| C-index | 0.66 |

The NRI of predictions using the proteomic model on the Whitehall II validation dataset was positive, and the results were better than the refit PCE model (developed on the HUNT3 training data) results on the HUNT3 verification data.

### Example 3. Cardiovascular Event Panel for Prediction of Secondary Cardiovascular Events

In order to predict the risk or likelihood that a subject with known apparently stable cardiovascular disease will have a secondary CV event within four years, a cardiovascular disease (CVD) secondary model containing a panel of 27 biomarker proteins was developed. The secondary CV event was defined as myocardial infarction, stroke, trans-ischemic attack, hospitalization for heart failure, or death. The training/verification analysis was developed using a sub-cohort of the HUNT3 data set, comprised of individuals that fulfilled eligibility criteria with known apparently stable CVD. The HUNT3 study includes 754 individuals with samples passing QC metrics, with 208 (28%) CV events observed within four years. The data were split into 80% for training and 20% for validation. The analysis was augmented by a 20% verification subset of the Atherosclerosis Risk in Communities (ARIC) visit 5 data set. (See The Atherosclerosis Risk in Communities (ARIC) Study: design and objectives. The ARIC investigators. Am. J. Epidemiol. 1989; 129(4):687-702.) Predictions in an independent replication set using the remaining 80% of the ARIC visit 5 and 20% of the HUNT3 data sets were unblinded during validation.

### Model

The cardiovascular disease (CVD) secondary model is an AFT parametric survival model with a Weibull distribution. This model has a panel of 27 biomarkers (log-10 and center-scaled) as features. A 4-year risk was reported. The predictions were binned into four risk bins and three risk categories. The Scoring and corresponding actual event rates in the training set over 4 years are reported in Table 7.

TABLE 7

| | | Minimum | Maximum | Observed | Proportion |
|---|---|---|---|---|---|
| Risk | | Risk | Risk | Event | to |
| Category | Risk Bin | Percentage | Percentage | Rate | Report |
| Low Risk | 1x | 0 | ≤7.5 | 5.13% | 5 in 100 |
| Medium | 1x-3x | >7.5 | ≤25.0 | 14.4% | 15 in 100 |
| Risk | 3x-6x | >25.0 | ≤50.0 | 36.4% | 35 in 100 |
| High Risk | 6x and | >50.0 | ≤100 | 64.6% | 65 in 100 |
| | above | | | | |

### Results

The C-index, AUC, and NRI relative to the refit PCE model (refit to the HUNT3 training data) for 4-year predictions are given in Table 8 below. The CVD secondary model had higher C-index and AUC values and a positive NRI at 4 years in the training (HUNT3) and verification (ARIC visit 5) datasets.

TABLE 8

| | | | | NRI (CVD |
|---|---|---|---|---|
| | | | | Secondary |
| Model | Dataset | AUC | C-index | vs. Refit PCE) |
| CVD Secondary | Training data | 0.797 | 0.766 | 0.350 |
| Model | Verification data | 0.795 | 0.728 | 0.821 |
| Refit PCE Model | Training data | 0.740 | 0.718 | N/A |
| | Verification data | 0.555 | 0.561 | N/A |

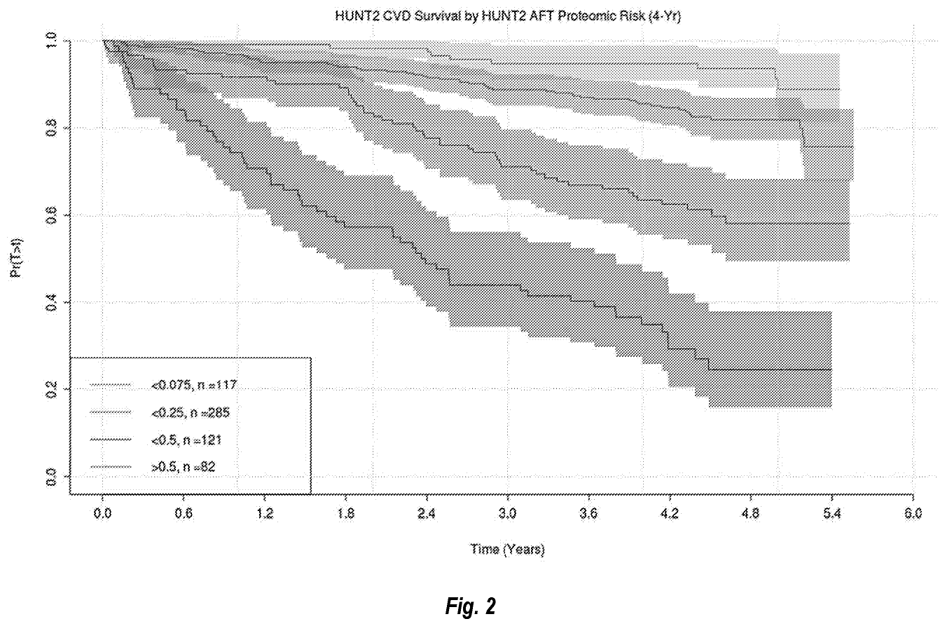

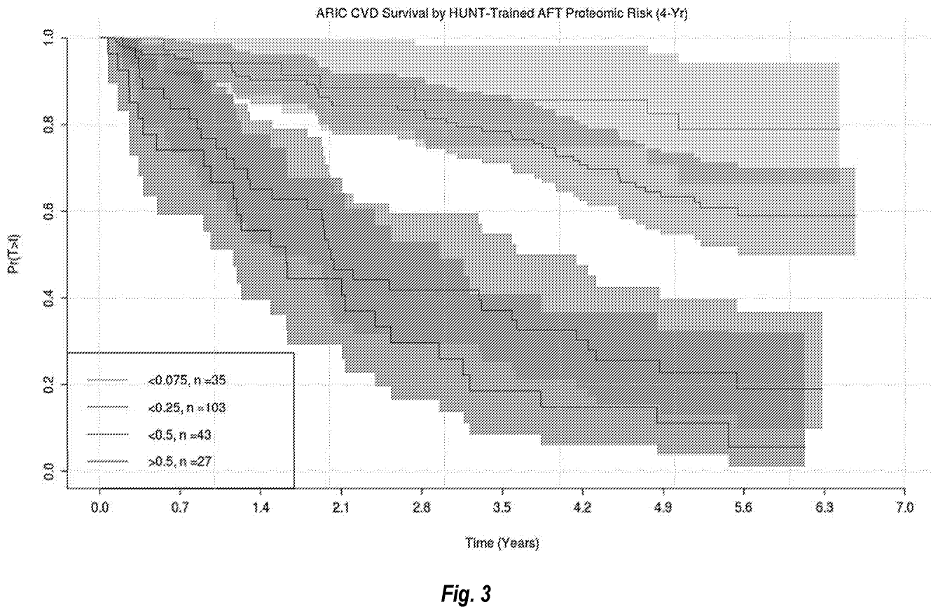

The risk probabilities of the training set were then categorized into 4 bins, such that the baseline group was relatively healthy (5% 4-year event rate), the high-risk group had a noticeably accelerated event rate (65% 4-year event rate), See Table 7 above. All 4 categories were distinct with 95% confidence at 4 years. The groups are shown in FIGS. 2 and 3 in the Kaplan-Meier survival curves, stratified by the four risk bins, for the HUNT3 training set (FIG. 2) and ARIC visit 5 verification set (FIG. 3). FIGS. 2 and 3 provide an overview of how the empirical distribution of CVD secondary events over time separates between the different predicted risk bin groups, with shaded regions representing 95% confidence intervals of the Kaplan-Meier estimates.

### Validation

Model validation was performed on the HUNT3 20% holdout and ARIC 80% holdout validation sets. The analyte RFU values in the dataset were log 10 transformed prior to analysis. Analyte RFU values were then centered and scaled based on distributions from the training set only, and out-of-range log 10 RFU values were imputed using the values calculated via winsorization. The C-index and AUV of the final CVD secondary model were evaluated on the dataset at the 4-year timepoint and compared to the refit PCE model. The NRI was calculated as a comparison to the refit PCE model, shown in Table 9.

TABLE 9

| | | | | NRI (CVD Secondary vs. |
|---|---|---|---|---|
| | | | | Refit PCE) [CVD Event |
| Model | Dataset | AUC | C-index | NRI, No-event NRI] |
| CVD Secondary | HUNT3 holdout | 0.77 | 0.73 | 0.60 [0.49, 0.12] |
| Model | ARIC visit 5 holdout | 0.74 | 0.70 | 0.53 [0.61, −0.08] |
| Refit PCE | HUNT3 holdout | 0.64 | 0.63 | N/A |
| Model | ARIC visit 5 holdout | 0.57 | 0.56 | N/A |

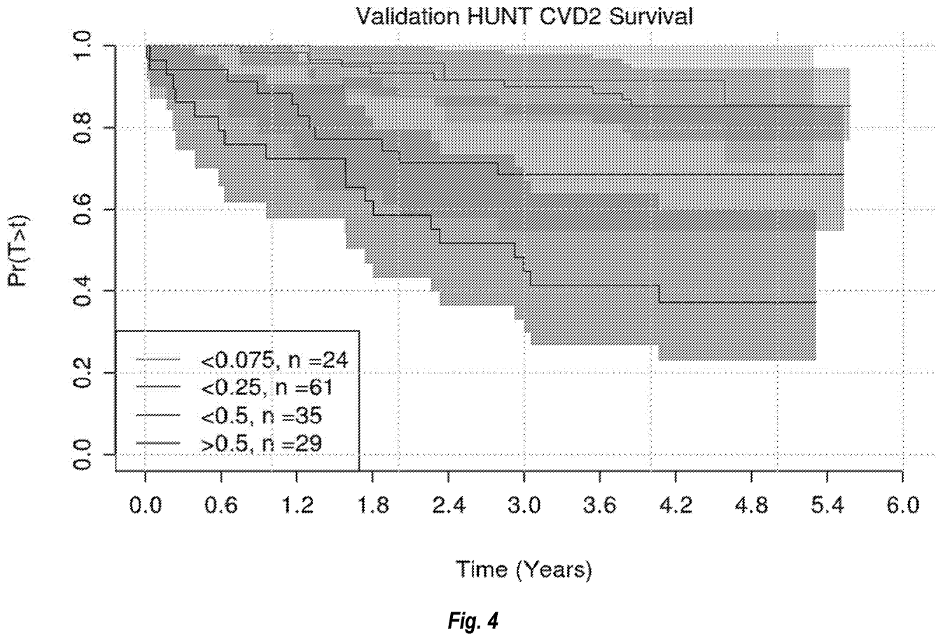

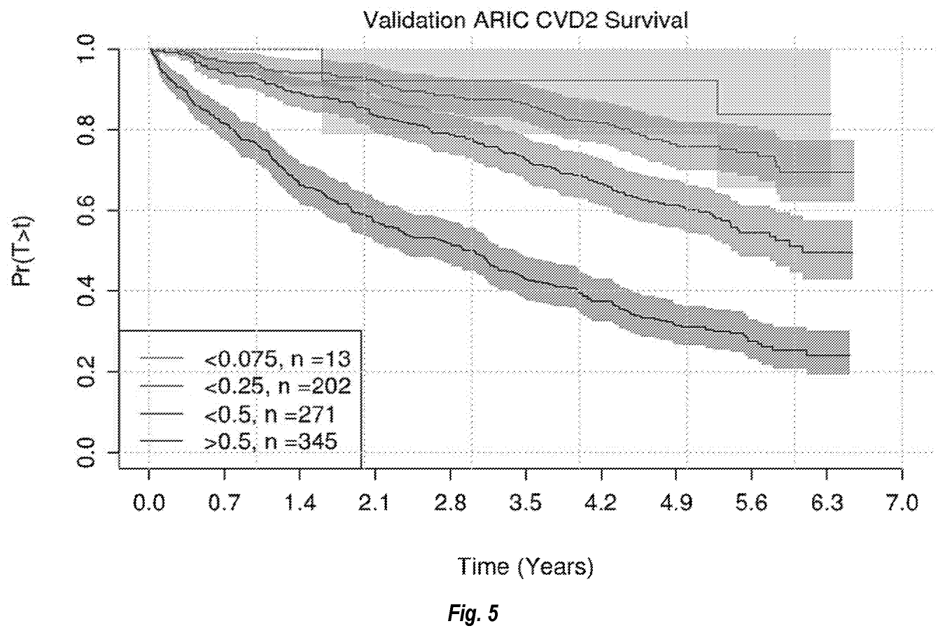

The CVD secondary model outperformed the refit PCE refit clinical model (using clinical and demographic parameters from the ACC risk equation, including separate coefficients by sex and ethnicity), in classifying 4-year events vs. no-event subjects, and concordance of predicting earlier events, in the training, verification, and validation sets. NRIs were positive for both validation sets (HUNT3 and ARIC visit 5). FIGS. 4 and 5 show the survival curves for the HUNT3 and ARIC visit 5 validation sets, stratified by cutoffs. The categorical ordering and slope were similar to the expected distribution from the training set, and empirically observed event rates. The highest-risk group was distinguished.

What is claimed is:

1. A method for screening a subject for the risk of a cardiovascular event (CV) event comprising:
(a) forming a biomarker panel comprising N protein biomarkers selected from N-terminal pro-BNP, sTREM-1, MMP-12, Antithrombin III, GPR56, Gelsolin, ST4S6, CHSTC, FSH, IL-1 sRII, PLXB2, SAP, and TFPI, wherein N is an integer from 3 to 13; and
(b) detecting the level of each of the N biomarkers of the panel in a sample from the subject.

2. A method for screening a subject for the risk of a cardiovascular event (CV) event comprising:
(a) forming a biomarker panel comprising N protein biomarkers selected from BNP, sTREM-1, MMP-12, SVEP1, ARL11, ANTR2, CA125, GOLM1, PPR1A, ERBB3, suPAR, GDF-11/8, JAM-B, ATS13, Spondin-1, NCAM-120, TFF3, SIRT2, ANP, NELL1, LRP11, NDST1, PTPRJ, CILP2, CA2D3, ITI heavy chain H2, and IGDC4, wherein N is an integer from 8 to 27; and
(b) detecting the level of each of the N biomarkers of the panel in a sample from the subject.

3. A method for predicting the likelihood that a subject will have a CV event comprising
(a) forming a biomarker panel comprising N protein biomarkers selected from BNP, sTREM-1, MMP-12, Antithrombin III, GPR56, Gelsolin, ST4S6, CHSTC, FSH, IL-1 sRII, PLXB2, SAP, and TFPI, wherein N is an integer from 3 to 13; and
(b) detecting the level of each of the N biomarkers of the panel in a sample from the subject.

4. A method for predicting the likelihood that a subject will have a CV event comprising
(a) forming a biomarker panel comprising N protein biomarkers selected from BNP, sTREM-1, MMP-12, SVEP1, ARL11, ANTR2, CA125, GOLM1, PPR1A, ERBB3, suPAR, GDF-11/8, JAM-B, ATS13, Spondin-1, NCAM-120, TFF3, SIRT2, ANP, NELL1, LRP11, NDST1, PTPRJ, CILP2, CA2D3, ITI heavy chain H2, and IGDC4, wherein N is an integer from 8 to 27; and
(b) detecting the level of each of the N biomarkers of the panel in a sample from the subject.

5. The method of claim 1 or 3, wherein N is at least 4, at least 5, at least 6, at least 7, at least 8, at least 9, at least 10, at least 11, at least 12, or 13.

6. The method of claim 2 or 4, wherein N is at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, or 27.

7. The method of any one of claims 1-6, wherein the risk or likelihood of the subject having a CV event within 4 years is high if the levels of at least 5, at least 6, at least 7, at least 8, at least 9, at least 10, at least 11, at least 12, or at least 13 biomarkers of the set of biomarkers are each abnormal relative to a control level of the respective biomarker.

8. The method of any one of claims 1-7, wherein the CV event is myocardial infarction, stroke, trans-ischemic attack, hospitalization for heart failure, or death.

9. The method of any one of claims 2, 4, or 6-8 wherein the subject has coronary artery disease.

10. The method of any one of claims 1, 3, 5, 7, or 8, wherein the subject does not have a history of CV events.

11. The method of any one of claims 2, 4, or 6-9, wherein the subject has had at least one CV event.

12. The method of any one of the preceding claims, wherein the sample is selected from a blood sample, a serum sample, a plasma sample, and a urine sample.

13. The method of claim 12, wherein the sample is a blood sample.

14. The method of any one of the preceding claims, wherein the method is performed in vitro.

15. The method of any one of claims 1, 3, 5, 7, 8, 10, or 12-14, wherein the method comprises contacting biomarkers of the sample from the subject with a set of capture reagents, wherein each capture reagent of the set of capture reagents specifically binds to a different biomarker being detected.

16. The method of any one of claims 2, 4, 6-9, or 11-14, wherein the method comprises contacting biomarkers of the sample from the subject with a set of capture reagents, wherein each capture reagent of the set of capture reagents specifically binds to a different biomarker being detected.

17. The method of claim 15 or 16, wherein each capture reagent is an antibody or an aptamer.

18. The method of claim 17, wherein each biomarker capture reagent is an aptamer.

19. The method of claim 18, wherein at least one aptamer is a slow off-rate aptamer.

20. The method of claim 19, wherein at least one slow off-rate aptamer comprises at least one, at least two, at least three, at least four, at least five, at least six, at least seven, at least eight, at least nine, or at least 10 nucleotides with modifications.

21. The method of claim 19 or claim 20, wherein each slow off-rate aptamer binds to its target protein with an off rate (t_{1/2}) of ≥30 minutes, ≥60 minutes, ≥90 minutes, ≥120 minutes, ≥150 minutes, ≥180 minutes, ≥210 minutes, or ≥240 minutes.

22. The method of any one of claims 15-21, wherein one capture reagent specifically binds to sTREM1.

23. The method of any one of claims 15-22, wherein one capture reagent specifically binds to MMP-12.

24. The method of any one of claims 15 or 17-23, wherein one capture reagent specifically binds to N-terminal pro-BNP.

25. The method of any one of claims 15 or 17-24, wherein one capture reagent specifically binds to Antithrombin III.

26. The method of any one of claims 15 or 17-25, wherein one capture reagent specifically binds to GPR56.

27. The method of any one of claims 15 or 17-26, wherein one capture reagent specifically binds to Gelsolin.

28. The method of any one of claims 15 or 17-27, wherein one capture reagent specifically binds to ST4S6.

29. The method of any one of claims 15 or 17-28, wherein one capture reagent specifically binds to CHSTC.

30. The method of any one of claims 15 or 17-29, wherein one capture reagent specifically binds to FSH.

31. The method of any one of claims 15 or 17-30, wherein one capture reagent specifically binds to IL-1 sRII.

32. The method of any one of claims 15 or 17-31, wherein one capture reagent specifically binds to PLXB2.

33. The method of any one of claims 15 or 17-32, wherein one capture reagent specifically binds to SAP.

34. The method of any one of claims 15 or 17-33, wherein one capture reagent specifically binds to TFPI.

35. The method of any one of claims 1-34, wherein the set of biomarkers comprises at least 13 biomarkers.

36. The method of any one of claims 1-34, wherein the set of biomarkers consists of 13 biomarkers.

37. The method of any one of claims 16-23, wherein one capture reagent specifically binds to SVEP1.

38. The method of any one of claims 16-23 or 37, wherein one capture reagent specifically binds to ARL11.

39. The method of any one of claims 16-23, 37, or 38, wherein one capture reagent specifically binds to ANTR2.

40. The method of any one of claims 16-23 or 37-39, wherein one capture reagent specifically binds to CA125.

41. The method of any one of claims 16-23 or 37-40, wherein one capture reagent specifically binds to GOLM1.

42. The method of any one of claims 16-23 or 37-41, wherein one capture reagent specifically binds to PPR1A.

43. The method of any one of claims 16-23 or 37-42, wherein one capture reagent specifically binds to ERBB3.

44. The method of any one of claims 16-23 or 37-43, wherein one capture reagent specifically binds to suPAR.

45. The method of any one of claims 16-23 or 37-44, wherein one capture reagent specifically binds to GDF-11/8.

46. The method of any one of claims 16-23 or 37-45, wherein one capture reagent specifically binds to JAM-B.

47. The method of any one of claims 16-23 or 37-46, wherein one capture reagent specifically binds to ATS13.

48. The method of any one of claims 16-23 or 37-47, wherein one capture reagent specifically binds to Spondin-1.

49. The method of any one of claims 16-23 or 37-48, wherein one capture reagent specifically binds to NCAM-120.

50. The method of any one of claims 16-23 or 37-49, wherein one capture reagent specifically binds to TFF3.

51. The method of any one of claims 16-23 or 37-50, wherein one capture reagent specifically binds to SIRT2.

52. The method of any one of claims 16-23 or 37-51, wherein one capture reagent specifically binds to ANP.

53. The method of any one of claims 16-23 or 37-52, wherein one capture reagent specifically binds to NELL1.

54. The method of any one of claims 16-23 or 37-53, wherein one capture reagent specifically binds to LRP11.

55. The method of any one of claims 16-23 or 37-54, wherein one capture reagent specifically binds to NDST1.

56. The method of any one of claims 16-23 or 37-55, wherein one capture reagent specifically binds to PTPRJ.

57. The method of any one of claims 16-23 or 37-56, wherein one capture reagent specifically binds to CILP2.

58. The method of any one of claims 16-23 or 37-57, wherein one capture reagent specifically binds CA2D3.

59. The method of any one of claims 16-23 or 37-58, wherein one capture reagent specifically binds to ITI heavy chain H2.

60. The method of any one of claims 16-23 or 37-59, wherein one capture reagent specifically binds to IGDC4.

61. The method of any one of claims 16-23 or 37-60, wherein one capture reagent specifically binds to BNP.

62. The method of any one of claims 2, 4, 6-9, 11-14, 16-23, or 37-61, wherein the set of biomarkers comprises at least 27 biomarkers.

63. The method of any one of claims 2, 4, 6-9, 11-14, 16-23, or 37-61, wherein the set of biomarkers consists of 27 biomarkers.

64. The method of any one of claims 2, 4, 6-9, 11-14, 16-23, or 37-63, wherein the subject has apparently stable cardiovascular disease.

65. The method of claim 64, wherein the apparently stable cardiovascular disease comprises a history of a myocardial infarction, stroke, heart failure, revascularization, abnormal stress test, imaging suggesting coronary heart disease, or abnormal coronary calcium score.

66. The method of claim 65, wherein the myocardial infarction or stroke occurred at least six months prior to the date the sample was taken from the subject.

67. The method of claim 65, wherein the abnormal stress test is a treadmill or nuclear medicine based test.

68. The method of claim 65, wherein the imaging suggesting coronary heart disease is an angiogram showing coronary artery stenosis of 50% or greater.

69. The method of any one of claims 1-68, wherein the subject is at least 40 years old.

70. The method of any one of claims 1-69, comprising determining the risk or likelihood of the subject having a cardiovascular event within four years from the date the sample was taken from the subject.

71. The method of claim 70, wherein the risk or likelihood of the subject having a cardiovascular event is within one, two, three, or four years from the date that sample was taken from the subject.

72. The method of claim 70 or 71, wherein the cardiovascular event is myocardial infarction, stroke, trans-ischemic attack, hospitalization for heart failure, or death due to cardiovascular disease.

73. The method of any one of claims 70-72, wherein the risk is determined as a quantitative probability.

74. The method of any one of claims 70-72, wherein the risk is determined as a qualitative level of risk.

75. The method of claim 74, wherein the qualitative level of risk is low, moderate, or high.

76. The method of any one of the preceding claims, wherein the risk or likelihood of a CV event is based on the biomarker levels and at least one item of additional biomedical information selected from
a) information corresponding to the presence of cardiovascular risk factors selected from the group consisting of prior myocardial infarction, angiographic evidence of greater than 50% stenosis in one or more coronary vessels, exercise-induced ischemia by treadmill or nuclear testing or prior coronary revascularization,
b) information corresponding to physical descriptors of the subject,
c) information corresponding to a change in weight of the subject,
d) information corresponding to the ethnicity of the subject,
e) information corresponding to the gender of the subject,
f) information corresponding to the subject's smoking history,
g) information corresponding to the subject's alcohol use history,
h) information corresponding to the subject's occupational history,
i) information corresponding to the subject's family history of cardiovascular disease or other circulatory system conditions,
j) information corresponding to the presence or absence in the subject of at least one genetic marker correlating with a higher risk of cardiovascular disease in the subject or a family member of the subject,
k) information corresponding to clinical symptoms of the subject,
l) information corresponding to other laboratory tests,
m) information corresponding to gene expression values of the subject, and
n) information corresponding to the subject's consumption of known cardiovascular risk factors such as diet high in saturated fats, high salt, high cholesterol,
o) information corresponding to the subject's imaging results obtained by techniques selected from the group consisting of electrocardiogram, echocardiography, carotid ultrasound for intima-media thickness, flow mediated dilation, pulse wave velocity, ankle-brachial index, stress echocardiography, myocardial perfusion imaging, coronary calcium by CT, high resolution CT angiography, MRI imaging, and other imaging modalities,
p) information regarding the subject's medications,
q) information corresponding to the age of the subject, and
r) information regarding the subject's kidney function.

77. The method of claim 76, wherein the at least one item of additional biomedical information is information corresponding to the age of the subject.

78. The method of any one of the preceding claims, wherein the method comprises determining the risk or likelihood of a CV event for the purpose of determining a medical insurance premium or life insurance premium.

79. The method of claim 78, wherein the method further comprises determining coverage or premium for medical insurance or life insurance.

80. The method of any one of claims 1 to 79, wherein the method further comprises using information resulting from the method to predict and/or manage the utilization of medical resources.

81. The method of any one of claims 1 to 80, wherein the method further comprises using information resulting from the method to enable a decision to acquire or purchase a medical practice, hospital, or company.
